(19) United States
(12) Patent Application Publication
(10) Pub. No.: US 2025/0265035 A1
(43) Pub. Date: Aug. 21, 2025

(54) INTERACTIVE MUSICAL INTERFACE TO ENHANCE FORMATIVE LISTENING EXPERIENCES FOR CHILDREN WITH AUTISM SPECTRUM DISORDER

(71) Applicants: Matthew Mauriello, Newark, DE (US); Daniel Stevens, Newark, DE (US); Elise Ruggiero, Newark, DE (US); Abhishek Karwankar, Newark, DE (US)

(72) Inventors: Matthew Mauriello, Newark, DE (US); Daniel Stevens, Newark, DE (US); Elise Ruggiero, Newark, DE (US); Abhishek Karwankar, Newark, DE (US)

(73) Assignee: University of Delaware, Newark, DE (US)

(21) Appl. No.: 19/201,395

(22) Filed: May 7, 2025

Related U.S. Application Data
(60) Provisional application No. 63/643,663, filed on May 7, 2024.
(60) Provisional application No. 63/800,898, filed on May 6, 2025.

(51) Int. Cl. G06F 3/16; A61B 5/00; G06F 3/04817; G06F 3/0482; G06F 3/0484; G06F 3/0487

(52) U.S. Cl. CPC G06F 3/165; A61B 5/4088; A61B 5/7475; G06F 3/04817; G06F 3/0482; G06F 3/0484; G06F 3/0487; A61B 2503/06

(57) ABSTRACT

An interactive audio control device having a user interface, the user interface having a plurality of selectable user input elements, each selectable user input element having an audio playback control corresponding to a predetermined audio layer of a selected multi-layer music track or a playback operation, wherein each selectable user input corresponding to a predetermined audio layer is configured to select from a plurality of playback modes corresponding to the predetermined audio layer and to provide a visual indication corresponding to the selected playback mode.

### CROSS-REFERENCE TO RELATED APPLICATIONS

This patent application is related to and claims the benefit of priority of U.S. Provisional Application 63/643,663, filed on May 7, 2024, titled “INTERACTIVE MUSICAL INTERFACE TO ENHANCE FORMATIVE LISTENING EXPERIENCES FOR CHILDREN WITH ASD,” and U.S. Provisional Application Ser. No. 63/800,898, titled “AN INTERACTIVE DASHBOARD FOR DATA-DRIVEN THERAPEUTIC MUSIC COMPOSITION,” filed May 6, 2025, the entire contents of which are incorporated by reference.

### FIELD

Embodiments relate to apparatuses, methods, and systems configured to provide an interactive musical interface for evaluating musical interactivity of patients with Autism Spectrum Disorder. In particular, embodiments relate to an interactive audio control device having a user interface including a plurality of selectable user input elements, each selectable user input element being an audio playback control corresponding to a predetermined audio layer of a selected multi-layer musical track or a playback operation. Each selectable user input element corresponding to a predetermined audio layer being configured to select from a plurality of playback modes corresponding to the predetermined audio layer and to provide a visual indication corresponding to the selected playback mode.

### BACKGROUND

Engaging in formative music experiences plays a crucial role in children's cognitive, motor, emotional, and social development. Children with Autism Spectrum Disorder (ASD) have been shown to benefit from musical interventions, which can improve peer interactions and provide insights into their behavioral and emotional states. However, some children with ASD experience sensory sensitivities, including adverse reactions to loud noises, high-pitched sounds, or certain timbres, which can make it difficult to enjoy music or participate in musical activities. These individuals may also exhibit variations in neural processing, complicating their involvement in musical activities that demand direction-following, like singing or playing instruments.

In 2020, the Centers for Disease Control and Prevention reported a significant rise in ASD diagnoses, affecting approximately 1 in 36 U.S. children. Music therapy, an evidence-based intervention, utilizes musical elements such as listening, singing, and playing along for individualized goals, aiding in communication and emotional regulation in children with ASD, as the structured nature of music is especially beneficial. Improving access to music experiences can improve therapeutic outcomes, considering the inconsistent availability of music therapy.

The present disclosure is directed to an interactive music-listening system that allows for evaluation (e.g., clinical evaluation) and data analysis of the music preferences of patients (e.g., people with ASD) to better inform clinicians and music composers, while providing an interactive audio controller that allows patients to customize their music-listening experience based on personal preferences. The system provides layer-based modification of multi-layer musical tracks, allowing patients to modify, via the interactive audio controller, each individual layer based on personal preferences for various characteristics of a musical composition, such as timbre, pitch, melody, harmony, tempo, etc., while tracking each interaction a patient has with a given multi-layer musical track and logging interactivity data to create interactivity data analysis tools for clinicians and composers to better understand the musical preferences of people with ASD.

### SUMMARY

Evaluation and data analysis of musical preferences and musical interactivity of people with ASD using systems and methods as described herein provides for a data-driven approach to understanding of the musical preferences of those with ASD, such that musical composers and/or clinicians can create and/or modify musical compositions tailored to those with ASD.

One aspect of the invention relates to an interactive audio control device. The interactive audio control device has a user interface having a plurality of selectable user input elements. Each selectable user input element is an audio playback control corresponding to a predetermined audio layer of a selected multi-layer musical track, or a playback operation. Each selectable user input element corresponding to a predetermined audio layer is configured to select from a plurality of playback modes corresponding to the predetermined audio layer and to provide a visual indication corresponding to the selected playback mode.

In some embodiments, the interactive audio control device has a housing, the user interface is a keypad disposed within the housing, and each of at least a subset of the plurality of selectable user input elements is a depressible button disposed on the keypad.

In some embodiments, each depressible button corresponding to a predetermined audio layer may be configured to permit a device user to sequentially step through the plurality of playback modes by repeatedly pressing the depressible button.

In some embodiments, the visual indication may be a different color corresponding to the selected playback mode.

In some embodiments, each depressible button has a plurality of LEDs disposed beneath a translucent cover, and the different color corresponds to illumination of a different LED or different combination of LEDs corresponding to each of the selected playback modes.

In some embodiments, the interactive audio control device may have a receiver for receiving audio layer playback mode data and a transmitter for transmitting audio playback control output data. The audio playback control output data may include one or more device user inputs.

Another aspect of the invention relates to a system for evaluating interactivity of a device user with music audio during an evaluation session. The system includes the interactive audio control device, one or more audio output devices, at least one device monitor-controller (DMC), and at least one processor. The DMC includes at least one memory configured to store data received from the transmitter of the interactive audio control device and an audio library having audio data for a plurality of multi-layer musical tracks, at least one DMC user interface, at least one display, and a communication link. The communication link is configured to exchange information with the interactive audio control device and the one or more audio output devices. The at least one processor is in communication with the communication link, the at least one memory, and the at least one display. The at least one processor is configured to generate, via the at least one display, a graphical user interface (GUI) that displays settings of the interactive audio control device, a plurality of device monitor-selectable user input elements, and a menu of the plurality of multi-layer musical tracks. The at least one processor is configured to receive, via at least one of the interactive audio control device via the communication link or via the at least one DMC user interface, one or more setting selection inputs. Each setting selection input includes a device user input form the interactive audio control device or a corresponding DMC user input form the DMC user interface. Each setting selection input is selected from the group consisting of: a multi-layer musical track selectin, a playback mode selection, and a playback operation selection. The at least one processor is configured to output audio layer playback mode data to the interactive audio control device via the communication link based on the received one or more setting selection inputs. The at least one processor is configured to transmit playback audio to the one or more audio output device via the communication link based on the received one or more setting selection inputs. The at least one processor is configured to log interactivity data corresponding to each setting selection input and store the interactivity in the at least one memory.

In some embodiments, the DMC user interface GUI may include an icon corresponding to each button of the interactive audio control device.

In some embodiments, each icon may have a plurality of display formats each corresponding to a corresponding visual indication of the interactive audio control device.

In some embodiments, each display format includes an icon color corresponding to the color of its corresponding button of the interactive audio control device, and providing the visual indication includes changing the color of one or more buttons and each corresponding icon based on the selected playback mode.

In some embodiments, each of the plurality of audio layers includes a plurality of playback modes including one or more distinct playback modes and a mute mode.

In some embodiments, the interactivity data includes timestamp data, audio layer data, and playback mode data.

In some embodiments, logging the interactivity data includes recording, in one or more data files, a timestamp for each setting selection input received by the at least one processor, a title of the multi-layer musical track being output as playback audio when each setting selection input is received, and the playback mode of each layer of the multi-layer musical track being output as playback audio when each selection setting input is received.

In some embodiments, the processor may be further configured to generate one or more visual data representations for evaluation. Each of the one or more visual data representations may include at least one of the interactivity data or the audio data.

Another aspect of the invention relates to a method for evaluating interactivity of a device user with music audio during an evaluation session. The method includes providing a system for evaluating interactivity of a device user with music during an evaluation session. The system includes an interactive audio control device having a user interface having a plurality of selectable user input elements. Each selectable user input element is an audio playback control corresponding to a predetermined audio layer of a selected multi-layer musical track, or a playback operation. Each selectable user input element corresponding to a predetermined audio layer is configured to select from a plurality of playback modes corresponding to the predetermined audio layer and to provide a visual indication corresponding to the selected playback mode. The system includes one or more audio output devices, at least one device monitor-controller (DMC), and at least one processor. The DMC includes at least one memory configured to store data received from the transmitter of the interactive audio control device and an audio library having audio data for a plurality of multi-layer musical tracks, at least one DMC user interface, at least one display, and a communication link. The communication link is configured to exchange information with the interactive audio control device and the one or more audio output devices. The at least one processor is in communication with the communication link, the at least one memory, and the at least one display. The method includes generating, by the at least one processor, via the at leas tone display, a graphical user interface (GUI) that displays setting of the interactive audio control device, a plurality of device monitor-selectable user input elements, and a menu of the plurality of multi-layer musical tracks. The method includes receiving, by the at least one processor, via at least one of the interactive audio control device via the communication link or the at least one DMC user interface, one or more setting selection inputs. Each setting selection input includes a device user input from the interactive audio control device or a corresponding DMC user input from the DMC user interface. Each setting selection input is selected from the group consisting of a multi-layer musical track selectin, a playback mode selection, and a playback operation selection. The method includes outputting, to the interactive audio control device via the communication link, audio layer playback mode data based on the received one or more setting selection inputs. The method includes transmitting, to the one or more audio output devices via the communication link, playback audio based on the receive one or more setting selection inputs. The method includes logging, by the at least one processor, interactivity data corresponding to each setting selection input. The method includes storing in the at least one memory, by the at least one processor, the interactivity data. The method includes generating, by the at least one processor, an evaluation output including at least one of the interactivity data or the audio data.

In some embodiments, the evaluation output may include one or more visual data representations each including at least one of the interactivity data or the audio data.

In some embodiments, the method further includes generating, by the at least one processor, a composer-interactivity interface including the one or more visual data representations.

Another aspect of the invention relates to a non-transitory computer memory programmed with computer-readable instructions for causing a computer to perform the method steps of: (i) generating, by at least one processor in communication with the non-transitory computer memory via a communication link, via at least one display, a graphical user interface (GUI) that displays settings of an interactive audio control device, a plurality of device monitor-selectable user input elements and a menu of a plurality of multi-layer musical tracks, each of the multi-layer musical tracks comprising a plurality of audio layers, each audio layer comprising a plurality of playback modes; (ii) receiving, by the at least one processor, via at least one of the interactive audio control device via the communication link or at least one device monitor-controller (DMC) user interface, one or more setting selection inputs, each setting selection input comprising a device user input from the interactive audio control device or a corresponding DMC user input from the DMC user interface, each setting selection input selected from the group consisting of: a multi-layer musical track selection, a playback mode selection, and a playback operation selection; (iii) outputting, by the at least one processor, to the interactive audio control device via the communication link, audio layer playback mode data based on the received one or more setting selections; (iv) transmitting, by the at least one processor, to one or more audio output devices in communication with the communication link, playback audio based on the received one or more setting selections; (v) logging, by the at least one processor, interactivity data corresponding to each setting selection input; (vi) storing in the non-transitory computer memory, by the at least one processor, the interactivity data; and (vii) generating, by the at least one processor, an evaluation output comprising at least one of the interactivity data or audio data for one or more of the plurality of multi-layered audio tracks.

In some embodiments, the evaluation output may include one or more visual data representations each including at least one of the interactivity data or the audio data.

Another aspect of the invention relates to a device monitor-controller (DMC) configured for communication with an interactive audio control device and one or more audio output devices. The DMC includes at least one memory configured to store data received from a transmitter of the interactive audio control device and an audio library having audio data for a plurality of multi-layer musical tracks, at least one DMC user interface, at least one display, a communication link, and at least one processor. The communication link is configured to exchange information with the interactive audio control device and the one or more audio output devices. The at least one processor is in communication with the communication link, the at least one memory, and the at least one display. The at least one processor is configured to generate, via the at least one display, a graphical user interface (GUI) that displays settings of the interactive audio control device, a plurality of device monitor-selectable user input elements, and a menu of the plurality of multi-layer musical tracks. The at least one processor is configured to receive, via at least one of the interactive audio control device via the communication link or via the at least one DMC user interface, one or more setting selection inputs. Each setting selection input includes a device user input form the interactive audio control device or a corresponding DMC user input form the DMC user interface. Each setting selection input is selected from the group consisting of: a multi-layer musical track selectin, a playback mode selection, and a playback operation selection. The at least one processor is configured to output audio layer playback mode data to the interactive audio control device via the communication link based on the received one or more setting selection inputs. The at least one processor is configured to transmit playback audio to the one or more audio output device via the communication link based on the received one or more setting selection inputs. The at least one processor is configured to log interactivity data corresponding to each setting selection input and store the interactivity in the at least one memory.

### BRIEF DESCRIPTION OF THE DRAWINGS

FIG. 1 illustrates an exemplary embodiment of an interactive audio control device.

FIG. 2 illustrates an exemplary embodiment of a device user selectable input element.

FIG. 3A illustrates an exemplary embodiment of system for evaluating interactivity of a device user with music audio during an evaluation session.

FIG. 3B illustrates an exemplary embodiment of a device monitor-controller.

FIG. 4 shows an exemplary embodiment of a graphical user interface.

FIG. 5 illustrates steps of an exemplary embodiment of a method for evaluating interactivity of a device user with music audio during an evaluation session.

FIG. 6 illustrates steps of an exemplary embodiment of a method for evaluating interactivity of a device user with music audio during an evaluation session.

FIG. 7 shows an exemplary embodiment of an interactive audio control device.

FIG. 8 shows an exemplary sensory room.

FIG. 9 illustrates a graphical representation of patient engagement time and standard deviation with each of four songs for a group of patients.

FIG. 10 illustrates a graphical representation of patient average interactions per song with standard deviation for a group of patients.

FIG. 11 illustrates a graphical representation of percentage of interactions by song and patient for a group of patients.

FIG. 12 illustrates a graphical representation of number and time of patient modifications to audio layers of “Twinkle, Twinkle Little Star.”

FIG. 13 illustrates a graphical representation of number and time of patient modifications to audio layers of “Row, Row, Row Your Boat.”

FIG. 14A illustrates a graphical representation of patient C3's modified version of “Ants go Marching.”

FIG. 14B illustrate a graphical representation of patient C5's modified version of “Ants go Marching.”

FIG. 15A illustrates a graphical representation of patient C3's modified version of “Wheels on the Bus.”

FIG. 15B illustrate a graphical representation of patient C5's modified version of “Wheels on the Bus.”

FIG. 16 illustrates an overview of steps of a clinical evaluation session.

FIG. 17 shows an exemplary interactive dashboard user interface.

FIG. 18 shows an exemplary interactive dashboard user interface.

FIG. 19 shows an exemplary interactive dashboard user interface.

FIG. 20 shows an exemplary interactive dashboard user interface.

FIG. 21 shows an exemplary interactive dashboard user interface.

### DETAILED DESCRIPTION

Objects, aspects, features, advantages and possible applications of the present innovation will be more apparent from the following description thereof, presented in conjunction with the drawings. Like reference numbers used in the drawings identify like components.

The following description of exemplary embodiments presently contemplated for carrying out aspects of the present invention is made merely for the purpose of describing general principles, examples, and features of embodiments of the present invention. The scope of the present invention is not limited to the embodiments as described in detail herein.

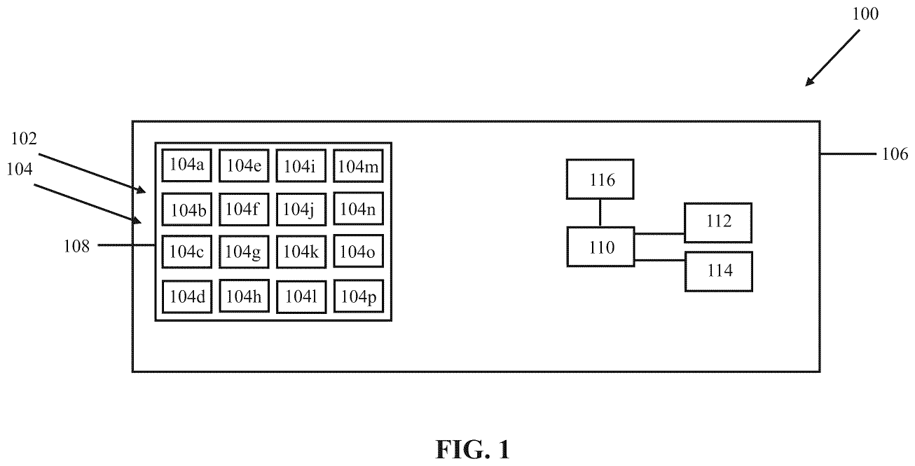

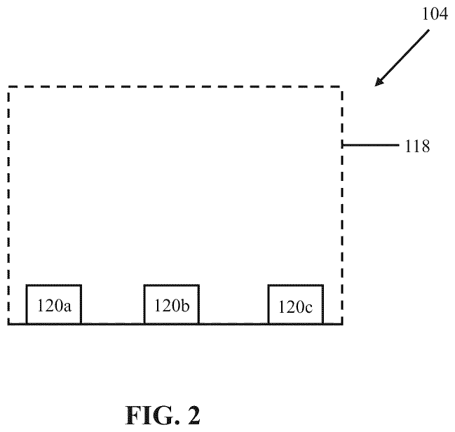

Referring to FIGS. 1 and 2, in an exemplary embodiment, audio control device 100 includes a user interface 102. User interface 102 includes a plurality of selectable user input elements 104 (e.g., selectable user input elements 104a-104p). Each selectable user input element may correspond to a predetermined audio layer of a selected multi-layer musical track or a playback operation. For example, in some embodiments, each of selectable user input elements 104a-104g may correspond to one predetermined audio layer of a multi-layer musical track having a plurality (e.g. seven) predetermined audio layers, and one or more of the remaining selectable user input elements 104h-104p may each correspond to a playback operation (e.g., play, pause, stop, skip to next musical track, skip to previous musical track, increase volume, decrease volume, etc.). In some embodiments, one or more of the selectable user input elements 104 may be configured to permit a device user to signal an emotional response. For example, one of the selectable user input elements 104 may correspond to a “like” or “dislike” emotional response. Each selectable user input element 104 corresponding to a predetermined audio layer may be configured to select from a plurality of playback modes corresponding to the predetermined audio layer and to provide a visual indication corresponding to the selected playback mode.

A multi-layer musical track may be a modular arrangement of a song, for example, a popular children's song (e.g., “Row, Row, Row, Your Boat,” “Twinkle, Twinkle Little Star,” etc.), containing a plurality of predetermined audio layers, with each audio layer designed to address diverse sensory and/or aesthetic goals of a clinical evaluation. For example, a multi-layer musical track may include a melody layer, a harmony layer, a countermelody or secondary melody layer, a bass line layer, a percussion layer, a bass drum layer, and an ambient sounds layer. Each audio layer may correspond to a plurality of distinct playback modes, with one or more distinct playback modes altering one or more musical characteristics of its corresponding layer, such as timbre, pitch, tempo, rhythm, harmony or harmonization, calmness, energy, etc., and one distinct playback mode being a mute mode configured to mute the audio of its corresponding audio layer.

For example, one or more layers and/or distinct playback modes may explore device user preferences for timbre and pitch range, one or more other layers and/or one or more distinct playback modes may investigate rhythmic and harmonic device user preferences, etc. It should be understood that the foregoing are merely exemplary predetermined audio layers, playback modes, and/or musical characteristics, and that a musical track may include any number of predetermined audio layers and/or corresponding playback modes suitable for exploring device user preferences for any relevant or suitable musical characteristic.

Each of the multi-layer musical tracks and their corresponding audio layers may be composed, arranged, mastered, etc., utilizing digital audio equipment and/or software, such as an FM and wavetable synthesizer (e.g., Nord Wave 2), a morphing analog synthesizer (e.g., PolyBrute®), a sampler and drum sequencer (e.g., Elektron Digitakt®), music notation software (e.g., Sibelius®, MuseScore®, etc.), a digital audio work station (e.g., ProTools®), etc. Each distinct playback mode corresponding to an audio layer may include any combination of one or more instruments, vocals, ambient sounds, etc., to create the desired audio layer playback mode. In some embodiments, each of the multi-layer musical tracks may be composed based on a preferred or predetermined musical characteristic to create an adaptable sensory experience for device users with neural processing sensitivities. For example, in some embodiments, one or more multi-layer musical tracks may be a known musical composition (e.g., popular children's song) composed at a slower tempo (as compared to the standard known tempo) to accommodate for neural processing sensitivities.

In some embodiments, the interactive audio control device includes a housing 106. The housing 106 may be any material (e.g., plastic, metal, etc.) suitable for housing components of the audio control device 100, such as electrical components, interactive components, etc. The housing 106 may be sized, shaped, etc. such that the audio control device 100 may be held and interacted with by a device user (e.g., a person/patient/child with ASD). For example, the material, shape, size, appearance, etc. of housing 106 may be based on the developmental stage of a device user or group of device users, e.g., the developmental stage of children with ASD, the developmental stage being representative of the cognitive and/or emotional capacity of the device user or group of device users.

In some embodiments, the user interface 102 may be a keyboard 102 disposed within the housing 106, and each of the selectable user input elements 104 may be a depressible button 104 disposed on the keyboard 102. The housing 106 may be an enclosure that defines an opening 108, and the keyboard 102 may be disposed within housing 106 enclosure and positioned within opening 108 such that each depressible button 104 may be accessed (e.g., pressed) by a device user, while additional components of the interactive audio control device 100 remain enclosed within the enclosure. In some embodiments, the interactive audio control device 100 may include an interactive display screen (e.g., a touch screen display) configured to provide virtual a plurality of virtual selectable user input elements (e.g., touch screen buttons) having the same or similar functionality of the depressible buttons 104. While not limited to any type of embodiment for the interactive buttons, it should be understood that in at least some embodiments, the presence of physical, depressible buttons may be important for certain users.

Each depressible button 104 corresponding to a predetermined audio layer may be configured to permit a device user to sequentially step, or cycle, through the plurality of playback modes corresponding to the predetermined audio layer by repeatedly pressing the depressible button. For example, selectable user input element or depressible button 104a may correspond to a melody audio layer of a multi-layer musical track, the melody audio layer having distinct playback modes M1, M2, M3, and Mute, and a device user may press the depressible button 104a to sequentially step from M1 to M2, M2 to M3, M3 to Mute, and so on and so forth.

In some embodiments, each selectable user input element 104 may include one or more visual indicators 120 (e.g., 120a-120c) configured to provide a visual indication to the device user in response to the device user selecting the selectable user input element 104. In some embodiments, the visual indication may be a predetermined color corresponding to a playback mode or a playback operation, each playback mode or playback operation corresponding to a different color. For example, in embodiments in which each selectable user input element 104 is a depressible button 104, each depressible button 104 may include a translucent or transparent cover 118 and one or more light emitting diodes (LEDs) 120 (e.g., 120a-120c) disposed beneath the cover 118. Each predetermined color may correspond to the illumination of a different LED 120 (e.g., 120a, 120b, or 120c) or a different combination of LEDs 120 (e.g., 120a, 120b, and 120c).

For example, utilizing the above example of the melody audio layer corresponding to depressible button 104a and having playback modes M1-M3 and Mute, LEDs 120 of depressible button 104a may be configured to illuminate the color red in response to the device user selecting playback mode M1, illuminate the color blue in response to the device user selecting playback mode M2, illuminate the color yellow in response to the device user selecting playback mode M3, and illuminate the color white in response to the device user selecting the Mute playback mode. In some embodiments, the LEDs 120 may be configured to blink or flash in synchronization with the beat, tempo, etc., of the audio layer corresponding to the depressible button 104.

In some embodiments, one or more of the depressible buttons 104 may each include a symbol or graphic disposed on the cover 118 to indicate or identify the depressible button's corresponding playback mode or playback operation. For example, a depressible button 104 corresponding to a melody audio layer may include a music note or series of music notes graphic, a depressible button 104 corresponding to a bass drum audio layer may include a drum graphic, a depressible button 104 corresponding to a play/pause playback operation may include a play/pause symbol graphic, etc.

In some embodiments, the interactive audio control device 100 may include at least one processor 110 disposed within the housing 106, a transmitter 112 disposed within the housing 106 and in connection/communication with the at least one processor 110, a receiver 114 disposed within the housing 106 and in connection/communication with the at least one processor 110, and one or more power supplies 116 in connection with, and configured to provide power to, the at least one processor 110. As used herein, it should be understood that reference to “a” or “at least one” processor or any processor as labeled and discussed herein includes embodiments in which more than one processor may be used in combination to perform the functions as described, without limitation. Likewise, reference to “a” or “at least one” memory or any computer memory or memory as labeled and discussed herein includes embodiments in which more than one computer memory may be used in combination to store the information as described.

In some embodiments, processor 110 may be a microcontroller (e.g., Arduino, ARM Cortex-M, etc.) configured to power and/or control the operation (e.g., illumination) of the LEDs 120 and to transmit and/or receive data (e.g., via the transmitter 112 and/or receiver 114, respectively) to and/or from a system (e.g., an external system) in communication with the interactive audio control device 100.

In some embodiments, the interactive audio control device 100 may include one or more transceivers configured to both transmit and receive data to and from a system in communication with the interactive audio control device 100. For example, the interactive audio control device 100 may include one or more transmitters 112, receivers 114, and/or transceivers configured to wirelessly communicate with an external system via one or more wireless protocols (e.g., Bluetooth, WiFi, Zigbee, etc.). In some embodiments, the one or more power supplies 116 may include one or more batteries, which may be rechargeable or non-rechargeable. Some embodiments may include a power supply that includes plug and associated wire for connecting to an electrical socket. Still others (e.g. one intended for use with rechargeable batteries) may have a plug-in mode, a battery-operated mode, and a charging mode, and may contain the various circuitry, including transformers and the like, as are known in the art for providing such utility.

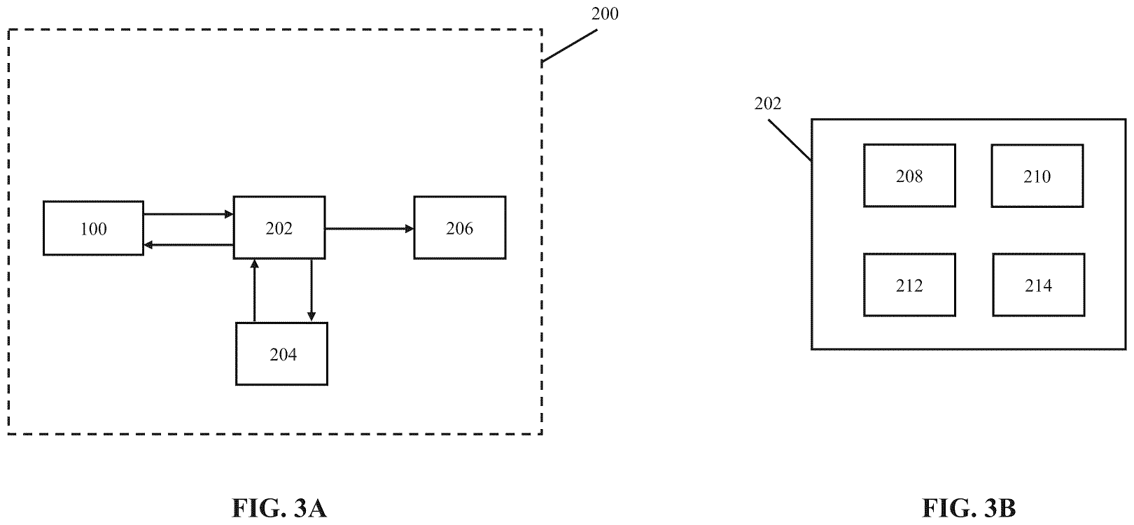

Referring to FIGS. 2-4, in an exemplary embodiment, a system 200 for evaluating (e.g., clinically evaluating) interactivity of a device user with music audio during an evaluation session includes the interactive audio control device 100, device monitor-controller (DMC) 202, at least one processor 204, and one or more audio output devices 206. The DMC 202 includes at least one memory 208 (e.g., a non-transitory computer memory) configured to store data received from the transmitter 112 of the interactive audio control device 100 and an audio library including audio data (e.g., wav, mp3, etc.) for a plurality of the multi-layer musical tracks, DMC user interface 210, at least one display 212, and a communication link 214 configured to exchange information with the interactive audio control device 100 and the one or more audio output devices 206. In use, typically one DMC 202 is paired with one audio output device 206 at a time, but it should be understood that exemplary systems may include multiple DMCs each capable of pairing with multiple devices. Processor 204 is in communication with communication link 214, memory 208, and display 212. In embodiments in which the interactive audio control device 100 includes processor 110, processor 110 may be in communication with the communication link 214 (e.g., via the transmitter 112 and/or receiver 114).

In some embodiments, the communication link 214 may include one or more transmitters, receivers, and/or transceivers configured to communicate, or exchange information (e.g., data) with components of the system 200 (e.g., the interactive audio control device 100, the one or more output devices 206, the at least one processor 204, etc.) via one or more wireless communication protocols (e.g., Bluetooth, WiFi, Zigbee, etc.). In some embodiments, the communication link 214 may be a physical connection (e.g., with data transfer/communication cables) to one or more of the components of the system 200.

In some embodiments, DMC 202 may be at least one computing device (e.g., a computer, tablet, smartphone, etc.) in communication with (e.g., via the communication link 214), or including, the at least one processor 204 and configured to run and/or host one or more software applications to provide the DMC user interface 210 to a DMC user such that the DMC user may enter, or provide, one or more DMC user inputs via the at least one DMC user interface 210. As is understood in the art, in embodiments, a local computing device may run an “app” comprising instructions executable by a local processor (e.g. on a laptop) to communicate with a remote processor (e.g. a server “in the cloud” accessible over a global communication network (i.e. the Internet) or accessible on a local network) to perform the functions attributed to the processor 204 as discussed herein. In some embodiments, DMC user interface 210 may include one or more interactive software applications having one or more graphical user interfaces (GUIs) that a DMC user may utilize to provide one or more DMC user inputs. For example, the DMC user interface 210 may include a Unity3D software application. It should be understood that the Unity3D software application is merely exemplary, and that the at least one DMC user interface 210 may include any type of software application suitable for providing the desired functionality of the at least one DMC user interface 210. The DMC 202 may include one or more input means configured to provide DMC user inputs, such as a touch screen, keyboard, mouse, etc. Thus, in embodiments, the user interface may be combined with the display, such as in a touchscreen operable device (e.g. a laptop, tablet, phone), whereas in other embodiments, a keyboard, mouse, or the like may comprise the user interface, whereas the display may be a computer display that displays, e.g., a cursor controllable by the mouse to make selections as described herein.

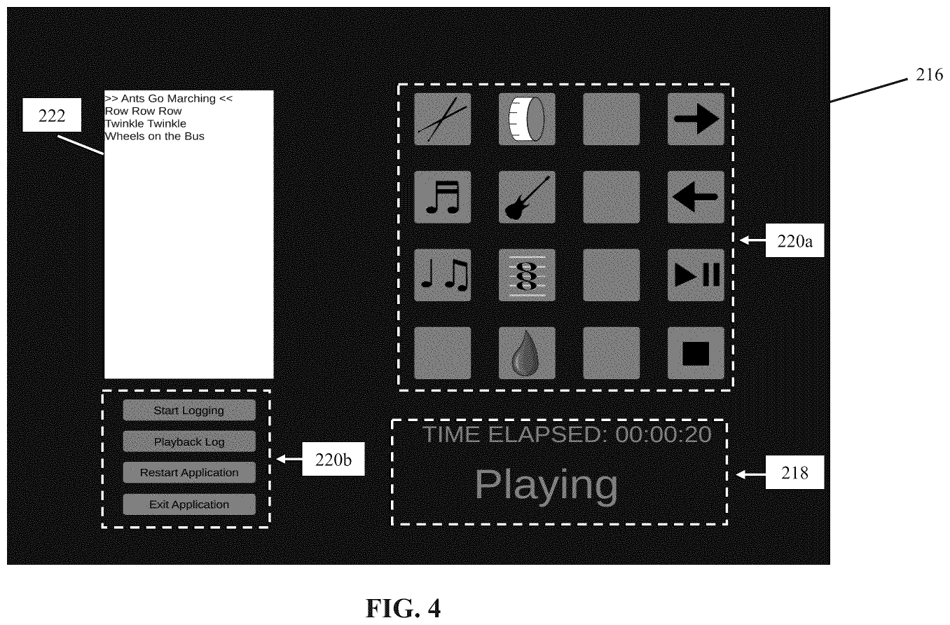

In some embodiments, processor 204 is configured to generate, via the display 212, a GUI 216 (FIG. 4) that displays settings 218 of the interactive audio control device 100, a plurality of device monitor-selectable user input elements 220 (e.g., 220a and/or 220b), and a menu of the plurality of multi-layer musical tracks 222. The processor 204 is configured to receive one or more setting selection inputs from the interactive audio control device 100 (e.g., via the communication link 214) and/or the DMC user interface 210. The setting selection inputs may include a device user input from the interactive audio control device 100 (e.g., a device user presses a button 104) and/or a corresponding DMC user input from the DMC user interface 210.

Referring to FIG. 4, the device monitor-selectable user input elements 220 may include icons 220, such as a plurality of icons 220a, at least a portion of which correspond to each device user selectable input elements 104 (e.g., depressible buttons 104), and one or more icons 220b, which may correspond to additional functions of the DMC 202 and/or the system 200, such as start logging, playback log, restart application, and exit application functions. The foregoing user input elements 220 are merely exemplary, and the GUI 216 may include any suitable user input elements 220 that provide a DMC user with control functionality of the DMC 202, the system 200, and/or elements/components thereof.

In some embodiments, each of the icons 220 corresponding to a button 104 may each include a plurality of display formats, each display format corresponding to a corresponding visual indication of the corresponding button 104. In some embodiments, each display format may include an icon color corresponding to the color of its corresponding button 104. In such embodiments, providing a visual indication (e.g., based on a selected playback mode) may include changing the color of one or more buttons 104 and changing the color each corresponding icon 220 to each correspond to the color corresponding to the selected playback mode. For example, if a device user or a DMC user selects, via the interactive audio control device 100 or the at least one DMC user interface 210, respectively, playback mode M1 (corresponding to the color red) for a given audio layer, the color of the button 104 and its corresponding icon 220 are changed, based on audio layer playback mode data transmitted from the at least one processor 110/204, to the color red.

In some embodiments, processor 204 may be configured to receive, e.g., via communication link 214, one or more setting selection inputs via the interactive audio control device 100 and/or DMC user interface 210. Exemplary setting selection inputs may include a device user input or DMC user input including one or more of a multi-layer musical track selection, a playback mode selection (e.g., an audio layer playback mode selection), and/or a playback operation selection (e.g., play, pause, start, stop, skip musical track, etc.). Processor 204 may be configured to output audio layer playback mode data to the interactive audio control device 100 (e.g., via communication link 214) based on the received one or more setting selection inputs.

For example, when processor 204 receives a setting selection input from the interactive audio control device 100 (e.g., the device user presses a button 104 that selects a playback mode for the audio layer corresponding to the button 104 pressed), the at least one processor 204 outputs, to the interactive audio control device 100, audio layer playback mode data based on the received setting selection input. Audio layer playback mode data may be data that describes or identifies the audio layer playback mode selected by pressing button 104, such that processor 110 of the interactive audio control device 100 changes the color of the button 104 to match the color corresponding to the selected audio layer playback mode (e.g., red for M1).

Similarly, for example, when the at least one processor 204 receives a setting selection input from the DMC user interface 210, e.g., the DMC user clicks or presses (e.g., on a touch screen) an icon 220 that corresponds to a button 104 corresponding to an audio layer, and the setting selection input selects a playback mode for the audio layer corresponding to the button 104, the at least one processor 204 outputs, to the interactive audio control device 100, audio layer playback mode data based on the received setting selection input. As described above, processor 204 may change the color of the icon 220 based on the selected audio layer playback mode, e.g., to match the color of the corresponding button 104.

In some embodiments, processor 204 may be configured to transmit playback audio (e.g., audio signals/data for one or more audio layers of a multi-layer musical track) to audio output devices 206 based on received setting selection inputs. In some embodiments, audio output devices 206 may be standalone audio output devices (e.g., speakers) in wireless or wired communication with processor 204. In some embodiments, the output devices 206 may be included in the interactive audio control device 100, the DMC 202, or any other component of the system 200 suitable for including one or more audio output devices 206.

In some embodiments, processor 204 may be configured to log interactivity data corresponding to each setting selection input received. For example, during an evaluation or session (e.g., clinical evaluation), processor 204 may log, or record, interactivity data for each device user input received from the interactive audio control device 100 and/or DMC 202. In some embodiments, the interactivity data may include timestamp data, such as a timestamp for each setting selection input received, the timestamp data representing the time at which the setting selection input was input by the device user and/or DMC user. In some embodiments, the interactivity data may include audio layer data, such as the title of the multi-layer musical track being output as playback audio when each setting selection input is received. In some embodiments, the interactivity data may include playback mode data, such as the current playback mode selected for each layer of the multi-layer musical track being output as playback audio when each setting selection input is received. In some embodiments, the interactivity data may include emotional response data, playback operational data, or any other data corresponding to device user or DMC user interactivity with the system 200 and/or components thereof.

In some embodiments, logging the interactivity data may occur throughout the entirety of an evaluation session: that is, the at least one processor 204 may be configured to automatically initiate or cease logging the interactivity data at the beginning or end, respectively, of the evaluation session, based on an operational occurrence, such as when playback audio is output for the first time, when playback audio output is complete, when the first setting selection input is received, etc. In some embodiments, the processor may be configured to initiate or cease logging the interactivity data based on a setting selection input corresponding to the logging function (e.g., a start logging icon 220 or depressible button 104).

In some embodiments, logging the interactivity data may include recording, by processor 204, in one or more data files (e.g., a .csv file), all or a portion of the interactivity data from an evaluation session. For example, an evaluation session (e.g., clinical evaluation session) may include a plurality of interactive audio control devices 100, each interactive audio control device 100 being interacted with by one device user (e.g., patient, child with autism, etc.), and one or more DMCs 202 each interacting with one or more DMC users (e.g., clinicians, parents, music composers, etc.), each DMC 202 corresponding to an interactive audio control device 100. In some embodiments, processor 204 may record all interactivity data for the evaluation session (e.g., each setting selection input received on each interactive audio control device 100 and/or DMC 202) in a single data file. In some embodiments, processor 204 may record all interactivity for each interactive audio control device 100 and/or each corresponding DMC 202 in a plurality of data files, each data file containing the interactivity data for one device user and corresponding DMC user(s).

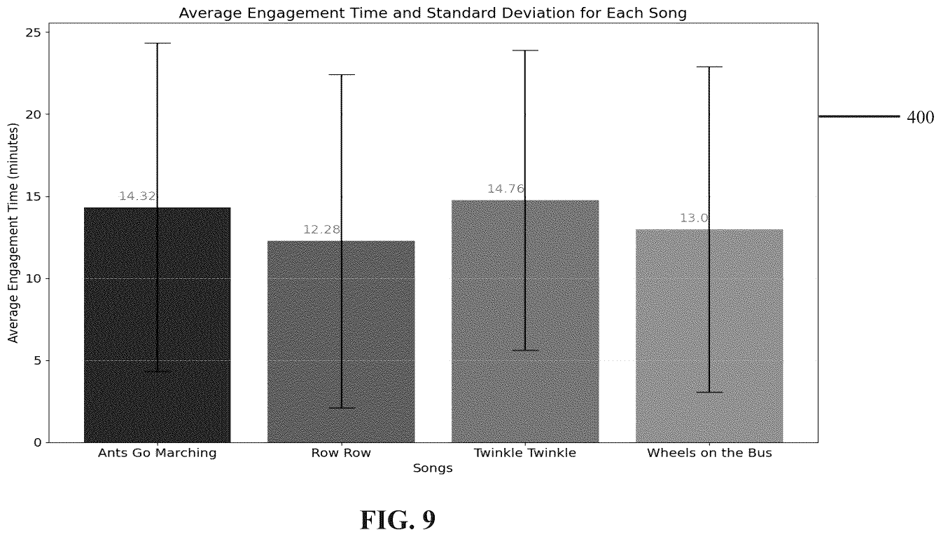

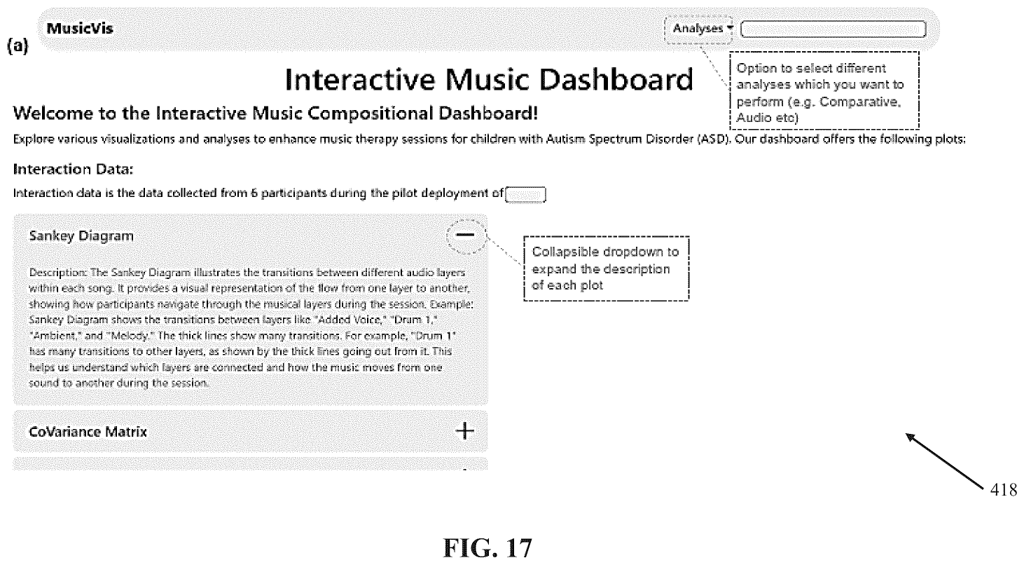

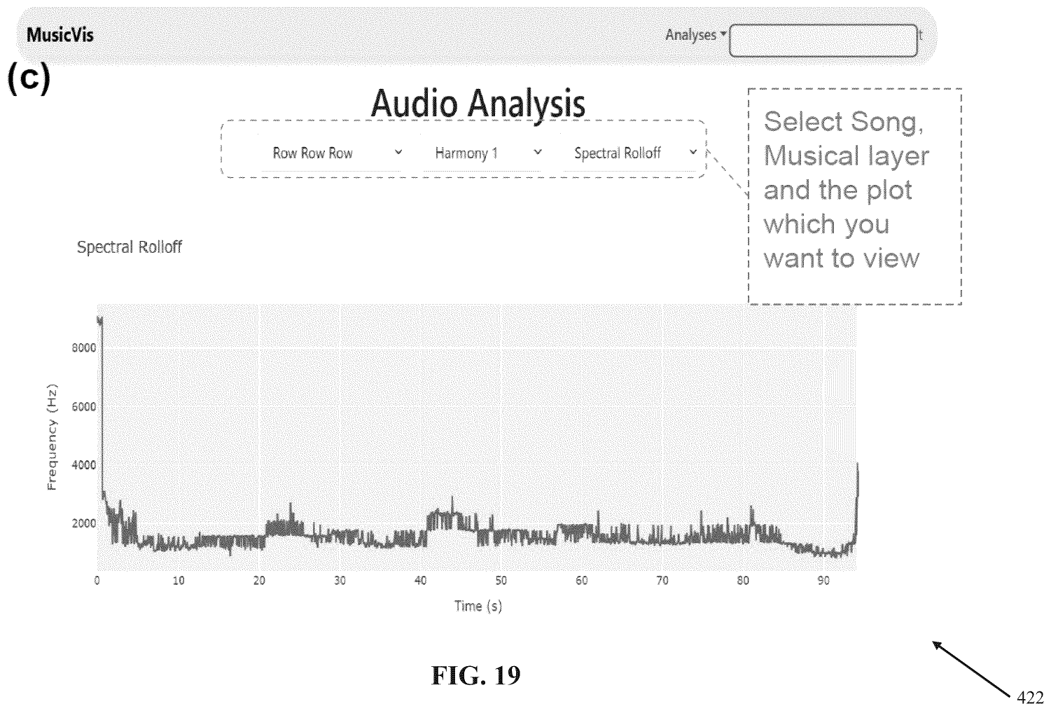

Referring now to FIGS. 9-21, in some embodiments, processor 204 may be configured to generate an evaluation output (e.g., clinical evaluation output) including one or more visual data representations (400-426) of the interactivity data recorded in the one or more data files (e.g., .csv files) and/or the audio data for each audio layer of each multi-layer musical track stored in one more data files (e.g., .wav files) for evaluation by a clinician, music composer, parent, etc. Exemplary visual data representations may include, but are not limited to, bar graphs (400, 402, 404, 424), line plots (408, 410), pie charts (410, 412, 414, 416), Sankey diagrams, CoVariance Matrices, waveplots (420), spectral centroids, spectrograms, spectral rolloffs (422), and Mel Frequency Cepstral Coefficients (MFCC).

For example, memory 208 may store one or more software applications, libraries, frameworks, neural networks, AI models, etc., and instructions (e.g., algorithms, program logic, etc.) that cause processor 204 to execute one or more desired functions, such as extracting the interactivity data and/or audio data from the one or more data files, processing the extracted data, parsing the extracted data, and/or generating one or more visual data representations based on the extracted and/or parsed data via the one or more software applications, libraries, etc. For example, memory 208 may include a software library (e.g., a plotly, plotly dash, Librosa, etc.) and instructions that cause processor 204 to interact with the software library to generate one or more visual data representations. In some embodiments, parsing the interactivity data may include parsing the interactivity data to identify audio layer transitions and/or time-based interactivity.

In some embodiments, processor 204 may be configured to generate one or more interactive interfaces, such as one or more interactive dashboards (e.g., 418-426), configured to allow interactive evaluation (e.g., evaluation by a clinician, music composer, parent, etc.) of the interactivity data and/or the audio data for each audio layer of each multi-layer musical track. For example, memory 208 may store one or more software libraries, applications, etc., (e.g., the dash application in the plotly dash library) configured to generate one or more interactive dashboards (e.g., a web-based dash interactive dashboard), the one or more interactive dashboards including one or more visual data representations. In an exemplary embodiment, processor 204 may be configured to generate one or more interactive dashboards designed specifically for interactive evaluation by a musical composer. For example, a composer-interactivity dashboard may include both interactivity data and audio data visual data representations. An interactive dashboard may include one or more interactive user interfaces (e.g., 418-426), that may each include one or more visual data representations and/or interactive user inputs that allow evaluators (e.g., clinicians, composers, etc.) to view, manipulate, edit, or otherwise interact with interactivity data, audio data, etc.

The above-described visual data representations and/or interactive dashboards may be stored in memory 208 to be accessed and analyzed by a clinician, composer, etc. after the evaluation session has concluded. In some embodiments, processor 208 may be configured to generate the visual data representations and/or interactive dashboards in real-time, e.g., during an evaluation session, and to display the visual data representations and/or interactive dashboards on one or more displays of system 200 (e.g., display 212) or one or more displays in communication with the system 200 (e.g., one or more remote displays in communication via the communication link 214).

In some embodiments, the system 200 may include one or more neural networks, generative AI models, machine learning models, etc., configured to determine device users' emotions, moods and/or musical preferences and to control one or more playback operations, in real-time, in response to the determined emotions, moods, and/or musical preferences. In such embodiments, the system 200 may include one or more video recording devices configured to transmit video footage of a device user during an evaluation session to be analyzed to determine a device users mood and/or emotional state. In some embodiments, the one or more neural networks, generative AI models, machine learning models, etc., may be configured to adjust one or more playback operations based on a real-time analysis of interactivity data. For example, if the system 200 determines that the device user has lost interest (e.g., ceases to interact with the interactive audio control device 100), the one or more neural networks, generative AI models, machine learning models, etc., may alter one or more aspects of the audio being heard by the device user, such as by changing one or more audio layers to a different playback mode, adjusting the volume of the music, altering the tempo of the music, etc.

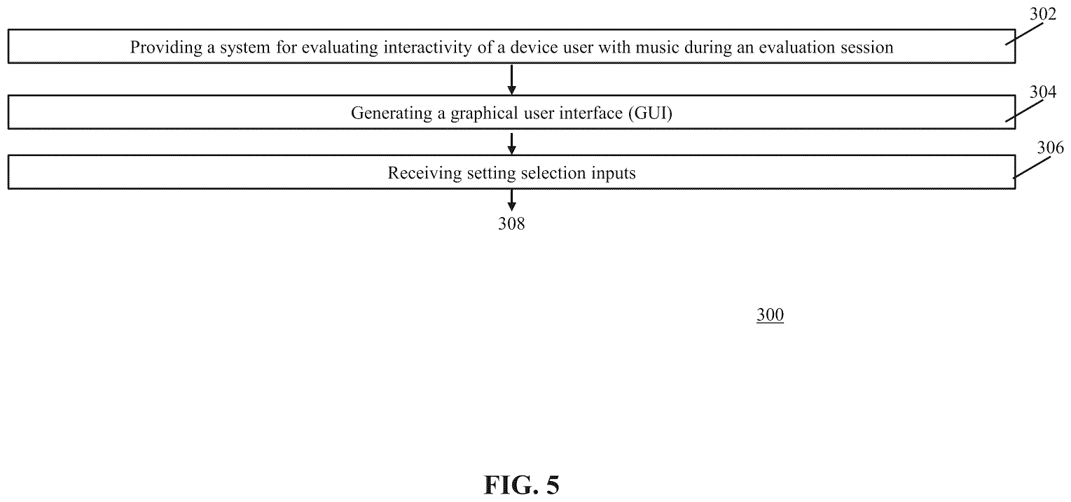

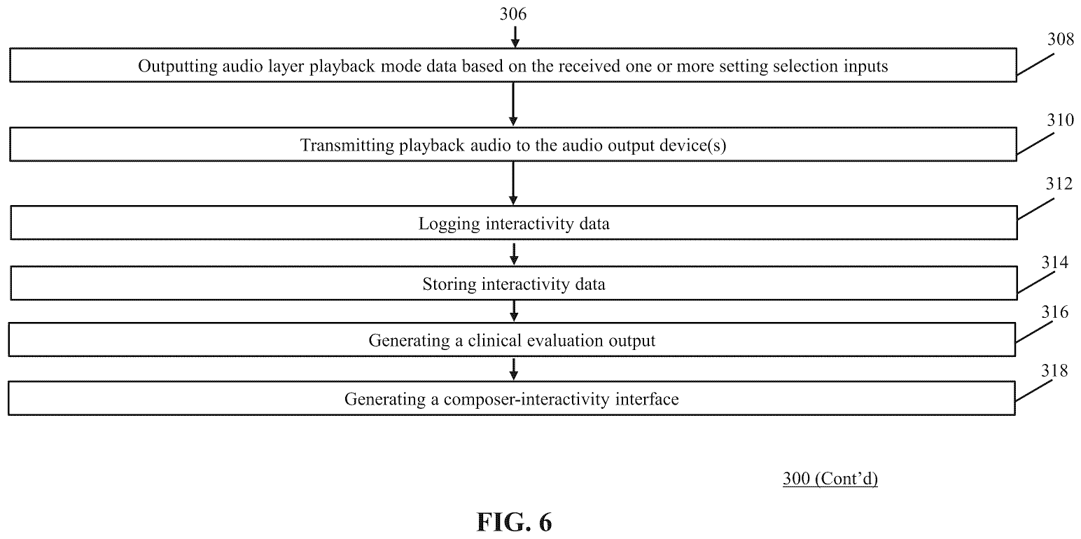

Referring to FIGS. 5 and 6, an exemplary method 300 for evaluating interactivity of a device user with music audio during an evaluation session is provided. The method 300 may utilize any of the above-described embodiments and/or elements of the interactive audio control device 100 and/or the system 200 for evaluating interactivity of a device user with music audio during an evaluation session, the details of which are not repeated here in the interest of brevity. The method 300 includes a first step 302, providing a system for evaluating interactivity of a device user with music audio during an evaluation session. The system may include an interactive audio control device including a plurality of selectable user input elements, each selectable user input element being an audio playback control corresponding to a predetermined audio layer of a selected multi-layer musical track or a playback operation. Each selectable user input element corresponding to a predetermined audio layer may be configured to select from a plurality of playback modes corresponding to the predetermined audio layer and to provide a visual indication corresponding to the selected playback mode.

The system may include one or more audio output devices. The system may include at least one device monitor-controller (DMC) including at least one memory configured to store data received from a transmitter of the interactive audio control device and an audio library comprising audio data for a plurality of multi-layer musical tracks. The system may include at least one DMC user interface, at least one display, and a communication link configured to exchange information with the interactive audio control device and the one or more audio output devices. The system may include at least one processor in communication with the communication link, the at least one memory, and the at least one display. Although discussed herein in an exemplary context of clinical evaluation of patients, such as patients with ASD, it should be understood that the invention is not limited to any particular purpose for using the system or characteristics of the users (of the audio control device or of the DMC). For example, a non-clinically-trained parent may be interested in monitoring a child (with or without ASD) using the system as described herein for any purposes whatsoever. Thus, the term “evaluation” may include non-clinical evaluation and monitoring using the system in addition to clinical evaluations as described in more detail herein.

The method 300 includes a second step 304, generating, by the processor, via the display, a graphical user interface (GUI) that displays settings of the interactive audio control device, a plurality of device monitor-selectable user input elements, and a menu of the plurality of multi-layer musical tracks. The method 300 includes a third step 306, receiving, by the at least one processor, via at least one of the interactive audio control device via the communication link or the at least one DMC user interface, one or more setting selection inputs, each setting selection input comprising a device user input from the interactive audio control device or a corresponding DMC user input from the DMC user interface, each setting selection input selected from the group consisting of: a multi-layer track selection, a playback mode selection, and a playback operation selection.

The method 300 includes a fourth step 308, outputting, to the interactive audio control device via the communication link, audio layer playback mode data based on the received one or more setting selection inputs. The method 300 includes a fifth step 310, transmitting, to the one or more audio output devices, via the communication link, playback audio based on the received one or more setting selection inputs. The method 300 includes a sixth step 312, logging, by the at least one processor, interactivity data corresponding to each setting selection input. The method 300 includes a seventh step 314, storing, in the at least one memory, the interactivity data. The method 300 includes an eighth step 316, generating, by the at least one processor, an evaluation output including at least one of the interactivity data or the audio data.

In some embodiments, the evaluation output may include one or more visual data representations, each of the visual data representations including at least one of the interactivity data or the audio data. In some embodiments, the method 300 may further include a ninth step 318, generating, by the at least one processor, a composer-interactivity interface including the one or more visual data representations.

Although the steps are shown in a numbered sequence herein, the method is not limited to any sequence of the method steps. Multiple iterations of the various steps may be performed in any order and may be repeated in a typical session involving use of the system in accordance with aspects of the method. While described herein with respect to the specific system as described herein, it should be understood that aspects of the method may be performed with systems having different components than those described herein. Also, although described in embodiments as integrated systems, it should be understood that aspects of the invention may also include each of individual components configured for use in such systems, and the hardware and software (e.g. machine-readable memories containing machine-readable instructions for causing a processor to execute method steps embodied in the instructions) associated with each component and its functionality as described.

In an exemplary embodiment, memory 208 is a non-transitory computer memory programmed with computer-readable instructions for causing a computer to perform one or more of the method steps of the method 300.

### EXAMPLES

The examples as discussed herein describe specific embodiments of the devices, systems, and methods as described herein as applied for assisting children, and more specifically children with ASD. However, it should be understood that other embodiments of the devices, systems, and methods as described herein are not limited to any particular use or to use by any specific type of user (child/adult/non-human animal) or with any specific diagnosed condition (e.g. ASD) or lack thereof.

### Example 1

### Introduction

Engaging in formative music experiences plays a crucial role in children's cognitive, motor, emotional, and social development. Children with Autism Spectrum Disorder (ASD) have been shown to benefit from musical interventions, which can improve peer interactions and provide insights into their behavioral and emotional states. However, some children with ASD experience sensory sensitivities, including adverse reactions to loud noises, high-pitched sounds, or certain timbres, Engaging in formative music experiences plays a crucial role in children's cognitive, motor, emotional, and social development. Children with Autism Spectrum Disorder (ASD) have been shown to benefit from musical interventions, which can improve peer interactions and provide insights into their behavioral and emotional states. However, some children with ASD experience sensory sensitivities, including adverse reactions to loud noises, high-pitched sounds, or certain timbres, that make it difficult to enjoy music or participate in musical activities. These individuals may also exhibit variations in neural processing, complicating their involvement in musical activities that demand direction-following, like singing or playing instruments.

In 2020, the Centers for Disease Control and Prevention reported a significant rise in ASD diagnoses, affecting 1 in 36 U.S. children. Music therapy, an evidence-based intervention, utilizes musical elements such as listening, singing, and playing along for individualized goals, aiding communication and emotional regulation in children with ASD as the structured nature of music is especially beneficial in reducing anxiety, enhancing social bonding, and facilitating self-expression, which are often challenges for children with ASD. Improving access to music experiences can improve therapeutic outcomes, considering the inconsistent availability of music therapy.

In this example, the inventors developed an interactive music system, uCue™, with a vision of—“You create, you enjoy”—tailored to children with ASD. uCue incorporates modular arrangements of children's songs set at an accessible tempo and allows users to modify musical layers as needed, similar to how a music conductor might cue a performer's entrance. As children use uCue to adapt musical sounds to their tastes, emotions, and expressive desires, the system collects data on listener interactions, including layer combination choices and timing, which can inform composers in creating more engaging music for this population. As early work, research questions were exploratory and focused on the potential impact of uCue, an interactive music system, for children with ASD. Specifically, we ask: Does interacting with uCue help children with ASD actively identify and express their musical preferences (RQ1)? Can engagement with uCue to create music facilitate emotional expression and enhance these children's enjoyment of music (RQ2)? And, how might children with ASD benefit from having greater autonomy over their listener experience (RQ3)?

Towards answering these questions, the inventors conducted a pilot study where uCue was deployed in listening sessions involving seven parent-child dyads across two phases of deployment at a local library's Sensory Room. Parent-child dyads were guided through interactions with uCue within the Sensory Room, followed by pre and post-session interviews to collect feedback. An analysis of our study data suggests that: (i) uCue's logs were able to gather valuable interaction data and provide insights about global musical preferences, including preferences and dislikes for specific musical styles, layer combinations, and energy levels, (ii) uCue supported personal expression, communication, and enjoyment through song modification, and (iii) independent use of uCue enabled children to exercise autonomy by selecting familiar songs and exploring various music combinations and emotional connections. These findings suggest that uCue has potential as an assistive, interactive musical device for children with ASD that can enhance access to formative and emotionally resonant musical experiences.

Among the contributions of this work included: (i) the design of a modular music template and a prototype music library (see Table 1), (ii) a prototype uCue music playback and logging system, (iii) empirical evidence suggesting uCue's potential to supports children with ASD in accessing formative music experiences, and (iv) insights into developing interactive music experiences with technology for children with ASD.

Children with ASD and the Value of Autonomy

The American Psychiatric Association characterizes ASD using two criteria: “Persistent deficits in social communication and social interaction across multiple contexts” and “Restricted, repetitive patterns of behavior, interests, or activities.” To address communication struggles, primary school educators prioritize social development of children with ASD, specifically targeting “interpersonal relationships, language acquisition, play, and other skills, and comorbid conditions such as cognitive deficits, physiological issues, and maladaptive behaviors.” While such children may have individual auditory sensitivities, they have been observed to generally “respond positively to activities involving music” and may exhibit unusually high musical competence and interest. Given the synthesis of repeating patterns and social communication common in musical experiences, musical interventions and therapies create ideal developmental opportunities for children with ASD.

Children's toy and book designers often prioritize autonomy, creating products that let children combine sounds to produce musical effects. Similarly, uCue enhances autonomy by enabling users to personalize the instrumentation of songs, partially taking on the roles of composer and performer. Unlike typical toys, uCue offers creative freedom using a high-quality sound environment, allowing users to shape music, control timing, and influence the piece's overall character. This autonomy expands a child's range of musical responses and empowers them to modify music for different outcomes.

### Exploring Musical Preferences & Sensitivities

The musical aptitude often observed in children with ASD was first noted by Leo Kanner in his seminal work on early infantile autism. Research shows that individuals with ASD may have superior aural discrimination compared to neurotypical individuals, as evidenced by a study which found enhanced pitch, interval discrimination, and memory in people with ASD. Another study suggested that children with ASD may have a more developed ability for aesthetic judgment of music, especially when exposed to classical music samples. These results fit within a larger body of work demonstrating heightened sensitivity to musical pitch and timbre by individuals with ASD, though there was more variability in the results of subjects with ASD than with their NT counterparts. Other notable auditory processing trends among listeners with ASD include the preference for familiar over environmental sounds, consonant over dissonant music, and non-vocal over vocal sounds. Previous work in music therapy tools for children with ASD often lacked the ability to adapt to individual auditory preferences and processing needs, limiting their effectiveness in addressing diverse user sensitivities. uCue addresses this limitation by enabling song customization, allowing children to add or remove sounds based on their likes or dislikes, while simultaneously logging these changes. This logged data facilitates opportunities to investigate global and individual preferences and sensitivities as well as further personalization.

### Music Therapy & Socio-Emotional Learning

Music is a promising tool for cultivating emotional awareness and regulation in individuals with ASD. Studies have demonstrated that emotion recognition in musical stimuli remains intact for some listeners with ASD, as evidenced by similar neural activity in emotion centers across control populations and high-functioning experimental participants with autism. Individuals with ASD often maintain intact emotion processing in music, providing a foundation for developing socio-emotional skills. This shared understanding of music can serve as a catalyst for forming friendships with peers. From an educational and therapeutic standpoint, these intact abilities offer support for improvements in expressing positive emotions, maintaining eye contact and initiating interaction, attuned movement, initiating turn-taking, and engaging in joint behavior.

Recent surveys indicate increasing evidence of the benefits of music therapy for children and adolescents with ASD. A multi-sensory approach within music sessions encourages individuals with ASD to engage more with the act of creating music, and with the therapists; in turn, this can increase their level of communication and social interaction beyond the sessions. uCue allows children with ASD to interact with music in a dynamic and customizable way, aligning auditory stimuli with other sensory inputs, making it potentially suitable to support the goals of music therapy including socio-emotional learning activities.

### Interactive Music Technology

Research in music technology designed for individuals with disabilities has been advancing for decades because such technology presents a transformative potential to create adaptive, interactive, and inclusive musical experiences As compared to known interactive musical devices for music therapy, uCue offers better portability, options for song personalization based on the needs of the child and their sound sensitivities, a musical library comprised of various children's songs specifically composed for children with ASD, and a robust logging system which can be used by educators and therapists to adjust the interventions.

### Audio Template and Music Library

In this section, we present our Music Library and sound design concept, further discuss the functionality of the music control interface, and outline fundamental design principles for creating musical layers.

uCue Music Library and Sound Design.

uCue's prototype modular music library included arrangements of four children's songs: “Twinkle, Twinkle Little Star,” “The Ants Go Marching,” “The Wheels on the Bus,” and “Row, Row, Row Your Boat.” These songs were suggested based on a discussion post about songs parents thought their children generally liked. Songs with the highest number of hits were prioritized in the music production process. The post also asked parents to share ambient sounds enjoyed by their children; these sounds were used when creating ambient sound layers for uCue. The songs were created using high-end digital audio equipment, including a Nord Wave 2 (FM and wavetable synthesizer), Arturia PolyBrute (morphing analog synthesizer), and an Elektron Digitakt (sampler and drum sequencer). The musical arrangements were composed using music notation software (Sibelius and MuseScore) and exported as MIDI data. This MIDI data was manipulated using a ProTools digital audio workstation (DAW), which was also employed to mix the final tracks. Several harmony tracks were recorded live using the Nord and Arturia synthesizers.

### Design Principles for Music Layers.

The modular music composition template includes fifteen tracks across seven layers, designed to test musical preferences (e.g., timbre, rhythm) with minimal tracks. While more tracks could provide additional options, they would complicate pattern identification and significantly increase composition time. To balance consistency, productivity, and research goals, we limited the template to fifteen tracks. Each musical layer is designed to address diverse sensory and aesthetic goals while aligning with research objectives. To create an adaptable sensory experience suitable for children with neural processing sensitivities, songs were composed at slower tempos, as recommended by our partner organization.

Supplemental layers with higher rhythmic activity ensure a sense of forward motion despite the slower pace. Each song features unique timbral, stylistic, and motivic elements, with distinctive acoustic profiles and virtual instruments. Songs include both calm and rhythmically active layers to examine contrasting preferences. Countermelodies enhance the main melody, and harmony layers balance calm and energetic variations, incorporating both common and unusual harmonizations. Table 1 (below) summarizes the layers and corresponding interface button colors. This structure allows 2,303 unique combinations per song, balancing variety with manageable workload.

uCue's Construction

The uCue controller was built using commercially available components following the Sparkfun 4×4 grid interface tutorial. The casing of the prototype was 3D printed and designed in SolidWorks11. It featured a two-part design, with a hollow box separated into upper and lower sections that fit together. The software was written in the Unity3D12 engine. The controller connects with the Unity3D application via a Bluetooth module. Internally, an Arduino was placed in the lower section powered by a 5V battery.

TABLE 1
An overview of the seven layers on the device. Colors that appear on the music-
control interface button when selected are shown under variant labels.

| | | Ants Go | Row, Row, Row | | Twinkle Twinkle |
|---|---|---|---|---|---|
| Layer | | Marching | Your Boat | Wheels on the Bus | Little Star |
| 1. Melody | M1 (red) | solo piccolo, | solo flute, middle | solo flute, | gentle plucked sound + |
| | | gentle attack, | register, reedy | high register, | sustained strings in |
| | | slow decay, very | timbre | traditional | high register |
| | | high register | | timbre | |
| | M2 (blue) | solo trumpet, | flute (melody) + | synthesized, | sharp, plucked sound |
| | | moderate attack, | harp (round) | warm lead | |
| | | no decay, middle | | sound, w/long | |
| | | register | | delay, reverb | |
| | M3 (yellow) | harp/choir hybrid, | harp/choir hybrid | hard lead sound | bass flute + synthesized |
| | | sharp attack, quick | (melody + round) | (snyth) + soft | high register reverb |
| | | delay (harp), long | | choir hybrid | |
| | | decay (choir) | | | |
| 2. Harmony | H1 (red) | pad (calm) | choir (calm) | string pad (warm, | pad (calm) |
| | | | | with bursts of | |
| | | | | bright sound) | |
| | H2 (blue) | synth keyboard | water glasses (calm, | synthesized, with | synthesized, with |
| | | (active) | with ringing higher- | repetitive gate and | repetitive gate and |
| | | | register sounds) | arpeggiations over | arpeggiations over |
| | | | | expansive register | expansive register |
| 3. Additional | A1 (red) | bell sound, high | harp + choir sound, | electric piano, | plucked guitar, |
| Melody | | register, large | lilting rhythm, | moves slowly | steady, moderate |
| | | ambitus, moderate | moderate speed, large | with melody | speed descant |
| | | speed | ambitus | | |
| | A2 (blue) | electric guitar, | solo oboe, small | synthesized bell | plucked guitar, |
| | | fast descant line | ambitus, motivic | with sine wave | faster triplet speed |
| | | | repetition, faster | decay, moderate | descant, inclusion of |
| | | | speed with uneven | speed, medium | triplets |
| | | | values | register | |
| | A3 (yellow) | synth hybrid sound, | synthesized guitar, | synthesized bell, | plucked guitar, |
| | | high register, | fast descant line, but | fast descant line, | fastest descant line |
| | | stylized, uneven | in lower register | high register | (2x speed of A1), |
| | | values (fast) | | | greater gestural |
| | | | | | variance |
| 4. Bass Line | B1 (red) | electric guitar, | synthesized plucked | plucked bass, | Deep lead synth sound, |
| | | simple part with | sound, arpeggiated | energetic but | melodically ornate |
| | | light rhythmic | texture built on bass | sticks on specific | walking-style bass |
| | | activity | line | pitches, repeating | |
| | | | | them | |
| | B2 (blue) | bass guitar, faster | string bass, 2:3 | plucked bass, | electric bass, chord |
| | | ornamentation | rhythm, walking bass | slower and mostly | roots doubled at the |
| | | with syncopation | pattern | melodic | octave, note |
| | | | | | repetitions create |
| | | | | | rhythmic activity |
| 5. Percussion | P1 (red) | trap set | trap + techno sounds | tom, hi-hat | tom, hi-hat, castanets |
| 6. Bass Drum | D1 (red) | synthesized bass | modified bass drum | sharp bass attack | sharp bass attack |
| | | hit sound | sound | with fast decay | with fast decay |

| | | |
|---|---|---|
| 7. Ambient Sounds | S1 (red) | flowing water/stream |
| | S2 (blue) | city traffic sounds |
| | S3 (yellow) | train crossing sounds |

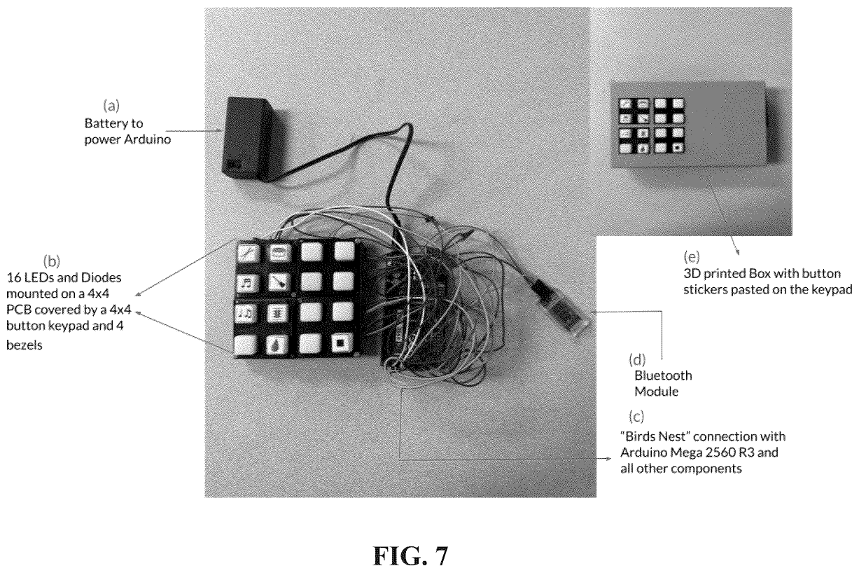

The PCB for the 4×4 grid button interface was positioned on top, aligning the interface buttons with the openings in the upper section of the case as illustrated in FIG. 7. Custom Arduino code was written to interface with our playback and logging software, where the playback software acted as an authoritative server, receiving input from the controller and sending back the updated state of the layers to be displayed on the controller's buttons using different colors.

### System Interactions & Control Interface

The seven audio layers are assigned their own music-control button on uCue. Each button is marked with a musical symbol indicating the layer it controls, and buttons change color to indicate which layer is active. Different playback modes are available for each audio layer. For example, four playback modes are available for the first audio layer: three unique instruments and a mute mode. After cycling through these modes on button presses, the layer reverts to its default configuration. This behavior is consistent across all seven audio layers, allowing users to manipulate each layer's playback mode using predefined interactions. uCue supports direct on/off transitions between different tracks and layers. The interface includes a vertical pane with four dynamically loaded song options, each with checkboxes for showing selection, and a central 4×4 color-coded button grid for controlling playback, navigating tracks, and managing musical layers. Playback controls include Play/Pause, Stop, and Forward/Backward buttons for navigating songs within the selected track. Status indicators at the bottom display “Playing,” elapsed time, and system controls like “Start Logging” and “Playback Log.” The “Start Logging” feature records all modifications to audio layers—whether via keyboard, click, or remote input—saving timestamps, layer names, and statuses in a .csv file.

### Method

Seven child participants were recruited across two phases, aged 4-12 years, with one exception of a 19-year-old adult child in a dyad, included upon parental request through email and social media outreach, direct verbal communication, and by seeking help from our community partners. Inclusion criteria required the presence of a parent or legal guardian during sessions, and children with auditory impairments were excluded. Participants were invited to complete a preliminary survey about their demographics and availability. Following this, the listening sessions were scheduled at the Sensory Room.

### Phase One Deployment

In the first phase, four parent-child dyads participated in a preliminary protocol. This included a parental consent and child assent process, a brief structured introduction interview, a listening session inviting children to interact with our music library using the uCue system in the Sensory Room, and a debrief interview.

### Phase Two Deployment

Based on feedback and observations, the preliminary protocol was updated to shorten the duration of pre- and post-interview to increase the time for challenges and include PECS (Picture Exchange Communication System) cards to engage more directly with non-verbal children. PECS cards were created for each of the four songs and the various musical layers available on the uCue interface. Additional cards allowed children to indicate their preferences (e.g., liking or disliking a particular song or layer) and express their emotions using ‘fill-in-the-blank’ cards designed to capture their mood during the session. This enhanced our interaction with a non-verbal participant. We recruited three additional parent-child dyads in this phase.

### Procedure

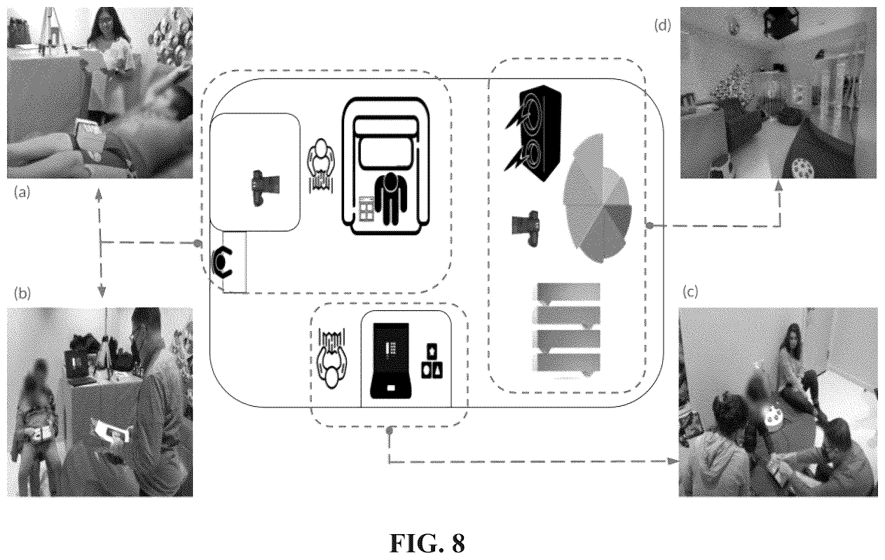

The study began by introducing the child to uCue, explaining the button functionality, and, for non-verbal participants, demonstrating the use of accompanying PECS cards to express reactions through emotion and fill-in-the-blank cards. FIG. 8 shows the arrangement in the sensory room and some of the images of the sessions conducted.

Once comfortable with uCue, child participants were asked to complete several challenges. In Challenge One, the participants used the device's [Play] and [Next Track] buttons to find a song they knew or liked. If necessary, cues for device functions and “liking” were provided using PECS cards. Following the song selection, participants were prompted to change the melody using the device, and their emotional responses to these changes were noted. Participants were prompted to use the buttons to explore the sound modifications and asked about specific sound changes they desired and if they wanted to try pressing buttons. They were also encouraged to adjust the song's dynamics, making it as calm or as energetic and loud as they preferred, by either verbally indicating their preferences or pointing to corresponding PECS cards. If the participant seemed disengaged at any point, they were prompted to express what they would like to hear with the use of examples and were given directions on how to achieve that. Participants were asked to reflect on their experience of modifying the song and what they thought and felt while pressing the buttons.

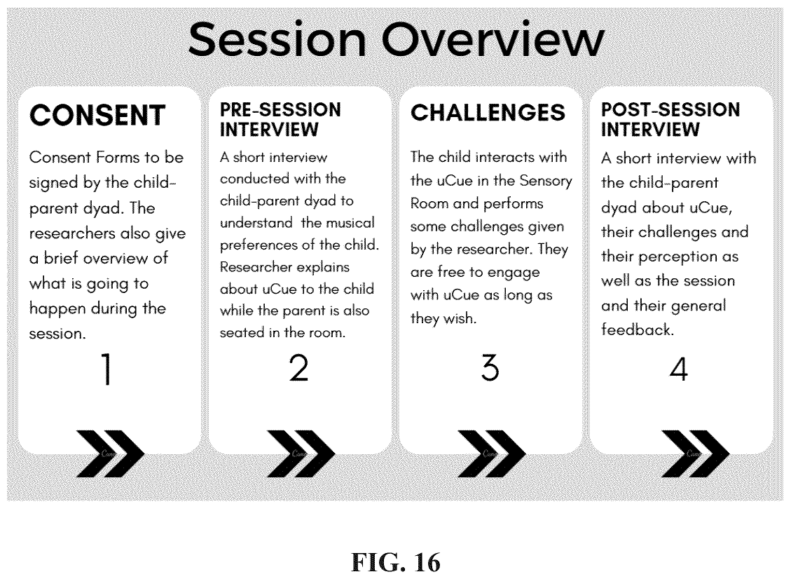

In Challenge Two, participants were prompted to choose a new track and modify it until they liked it. Participants shared thoughts as they listened to their customized song. Participants were given the opportunity to make further changes to the song if desired. To determine whether the participant was enjoying the song, they would be prompted to confirm their preference and in the absence of a response, rely on observational evidence from the session. They were also asked if they wanted to engage in singing or dancing while listening, and if the reaction to this was positive, the team would encourage them to do so by either cheering or joining in. If they seemed enthusiastic about engaging further, they were introduced to challenge three. In Challenge Three, if the child was capable of speaking or singing, they were encouraged to find a song they knew and liked, choose the sounds they preferred, and attempt to sing along with the music, either independently or with a parent. If they were not capable of singing, they were encouraged to dance, move, or clap along. These rounds assessed their listening skills, ability to control music through uCue, and emotional connection to the tracks. During the session, they were also allowed to interact with other objects in the sensory room. FIG. 16 provides an overview of the study, illustrating the steps followed during the session.

### Data Analysis

Analysis of collected data focused on understanding the child participants' music preferences, attention levels, and emotional responses, as well as their interactions with the device and music. Sources included uCue's system application logs, session audio/video recordings, and observations made by our research team during the session. A mixed-method approach, combining qualitative video analysis with a checklist and quantitative analysis of system logs, was employed. Key metrics' mean, median, and standard deviation were calculated and visualized through stacked bar plots. The video was analyzed using a structured checklist to assess participant actions and behaviors. A researcher team reviewed the videos, and participants' responses were tagged based on actions the research team observed. Due to the small sample size and early nature of the work, Inter-Rater Reliability (IRR) was not applied; instead, coding consistency was ensured through regular team meetings and cross-checking. Members of each parent-child dyad are referenced using P# and C#, respectively, with “#” representing the session number in our analysis. C7 was excluded from the logged data dataset due to an error where no data was recorded. Additionally, data from the beginning of the session, which involved introducing uCue to the child to the start of the first challenge, was excluded to ensure consistency in identifying their preferred sound choices.

### Results

### Pre-Session Interview Results

In a pre-session, parents and children were interviewed about the children's prior experiences with music.

### Music Interest and Influences

Parents and children agreed that the children enjoyed a wide range of music, from early child songs to specific genres like Jazz, Louisiana, brass, and more complex compositions like “Pluto's Reprisal.” Some expressed a strong love for music, while others were more reserved or even disliked certain genres. C3's statement highlighted this range of preferences, “I tend to like a lot of different songs of different genres.” A few participants (2/7), highlighted a preference for music with a clear sound and a single dominant instrument. For example, P5, noted that their child “ . . . likes when there's a clear sound, one instrument that comes through [in a song].” Their preferences were possibly shaped by their family environment and cultural background. C7 mentioned enjoying Spanish music due to his mother's influence, emphasizing the bonding experience, “I listen to Spanish music because my mom plays it in the car. It's fun to share that experience with her.”

### Listening Habits

The listening habits of the children were diverse. Differences in the children's responses, such as suddenly losing interest or requesting to turn off music, stand out as distinct from what might be expected in neurotypical children. As explained by P3: “Sometimes he will listen to it, and then at the end, he'll be like, okay, turn it off, because I think it's just too much.” Some often engaged with music in specific contexts, such as in the car or playing songs at home. One child, C7, mentioned a regular music routine before bedtime, indicating a consistent pattern in their music consumption. C3 enjoyed listening to familiar music (such as child-friendly theme songs from shows, Disney films, or folk songs like “The Wheels on the Bus”). Our in-session observations further support these variations, highlighting the unique reactions and attention patterns exhibited by children with ASD.

Other Interactions with Music

Parents detailed a spectrum of behaviors in how their children respond to music with common expressions of enjoyment like making sounds or moving. P1 pointed out their child's discomfort with particular sounds, stating, “There are sounds that he really responds negatively to He doesn't like it.” P2 noted their child's difficulty in imitating melodies, saying, “He can't imitate the sounds in the songs. He's not able to say things like ‘Moo’ or make other sounds” and also talked about their child's curiosity as, “If he's not sure what song it is, I think he becomes more attentive (P2).” Dislikes for certain songs or genres were expressed vocally or by attempts to change the track. However, physical engagement including activities such as singing, clapping, dancing, and tapping to express enjoyment of music was common among the children. For example, C7 said, “I can feel the rhythm in my body, and it makes me want to move. Faster tempos and energetic beats really catch my attention.” Likewise, P1 shared their child's enthusiasm for music saying, “He loves to sing and dance, singing the songs he knows, and he likes to roll on his belly when we sing ‘Head, shoulders, Knees, and Toes’, he is happy to have me sort of move his hands around.”

### Session Results

We analyzed six participants' application logs to understand their music preferences in the session. We coded their reactions and engagement with uCue by reviewing the video recordings. The quantitative analysis is presented alongside qualitative observations.

### Global Music Preferences

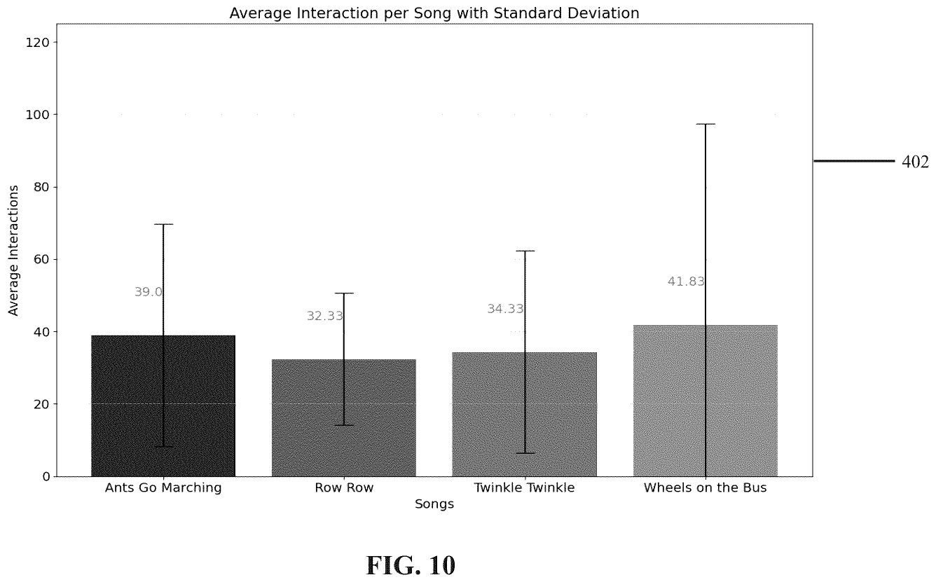

Analyzing children participants' music preferences included identifying sections of the session where children were adapting music intentionally (post-familiarization). This included evaluation of the total duration each song and each layer played during these intentional interaction periods. Children's prolonged engagement with specific songs and layers, as indicated by play length, was interpreted as a sign of preference. Verbal and non-verbal behaviors during the sessions provided further evidence of children's preferences. The observations made in Table 3 (below) highlight a range of engagement behaviors that reflect participant responses during the sessions. Results indicated that music preferences varied in sessions with uCue. Particularly, “Ants Go Marching” and “Twinkle, Twinkle” emerged as favorites. Most participants (4/7, 57.14%) favored these over other songs in the playlist based on play length, corroborated by feedback during the session and post-session interviews. FIGS. 9 and 10 show the duration and interaction for each song across participants, respectively.

TABLE 3

| Actions/Sessions | C1 | C2 | C3 | C4 | C5 | C6 | C7 | Total Count |
|---|---|---|---|---|---|---|---|---|
| Enthusiastically React to Music | ✓ | X | ✓ | X | ✓ | ✓ | ✓ | 5/7 (71.4%) |
| Stop Button Pressing for | ✓ | ✓ | X | X | ✓ | ✓ | ✓ | 5/7 (71.4%) |
| Preferred Sounds | | | | | | | | |
| Smile at Preferred Sound | ✓ | X | X | X | ✓ | ✓ | ✓ | 4/7 (57.14%) |
| Combinations | | | | | | | | |
| Engage with Prompts | ✓ | ✓ | ✓ | X | ✓ | ✓ | ✓ | 6/7 (85.71%) |
| Exhibit Relaxation | ✓ | X | ✓ | X | ✓ | ✓ | ✓ | 5/7 (71.4%) |
| Exhibit Energy | ✓ | ✓ | ✓ | X | ✓ | ✓ | ✓ | 6/7 (85.71%) |
| Noticeable Mood Change | ✓ | ✓ | ✓ | ✓ | ✓ | ✓ | ✓ | 7/7 (100%) |
| Hum or Sing Along | X | X | ✓ | X | ✓ | ✓ | ✓ | 4/7 (57.14%) |
| Interact with PECS Card | X | X | X | X | ✓ | ✓ | ✓ | 3/7 (42.85%) |
| Dance, Rock, or Tap Along | ✓ | X | ✓ | X | ✓ | ✓ | ✓ | 5/7 (71.4%) |
| Total Count | 8/10 | 4/10 | 7/10 | 1/10 | 10/10 | 10/10 | 10/10 | |
| | (80%) | (40%) | (70%) | (10%) | (100%) | (100%) | (100%) | |

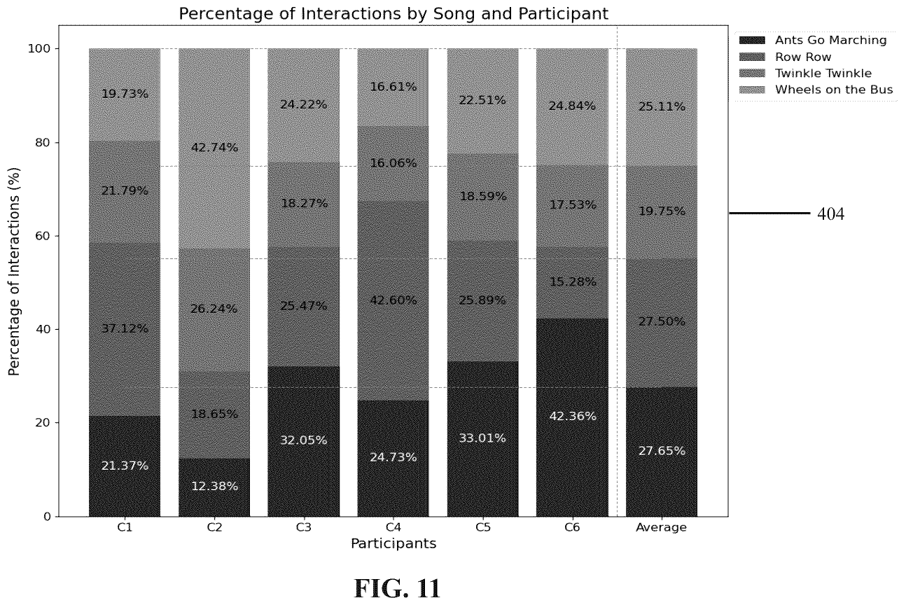

Observations suggest children display more enthusiasm for songs they listen to longer, indicating a preference for these tracks. Repeated interactions with the same song by different children support this view. Parent feedback confirms that some songs prompt more interaction because children genuinely enjoy them and react positively. FIG. 11 suggests that children have individual preferences for songs, with some songs resulting in more interactions.

### Response to the Prompts

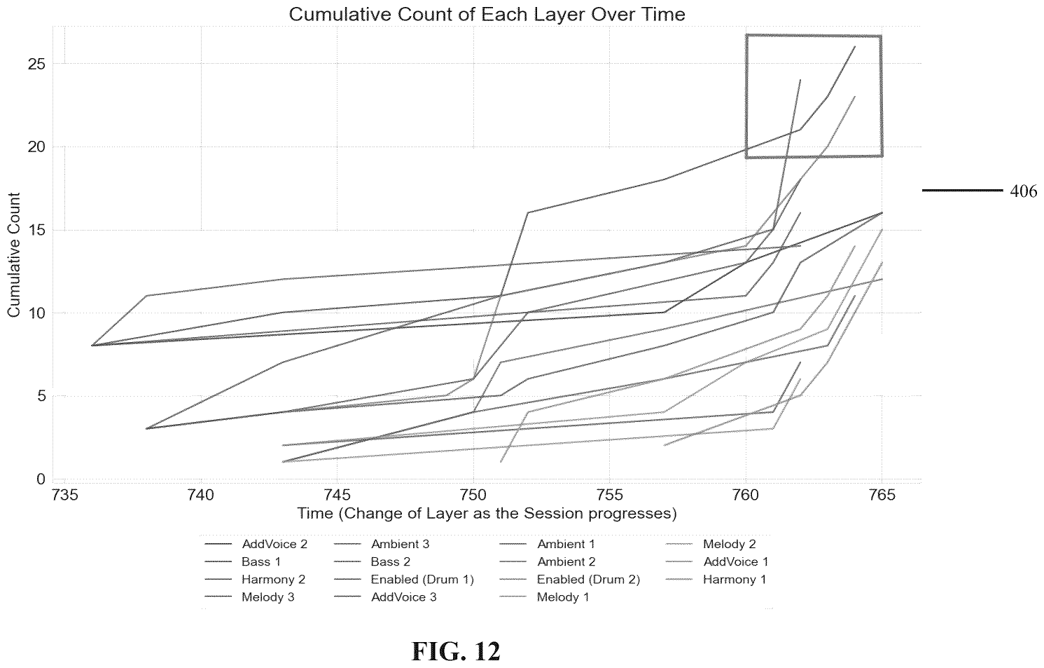

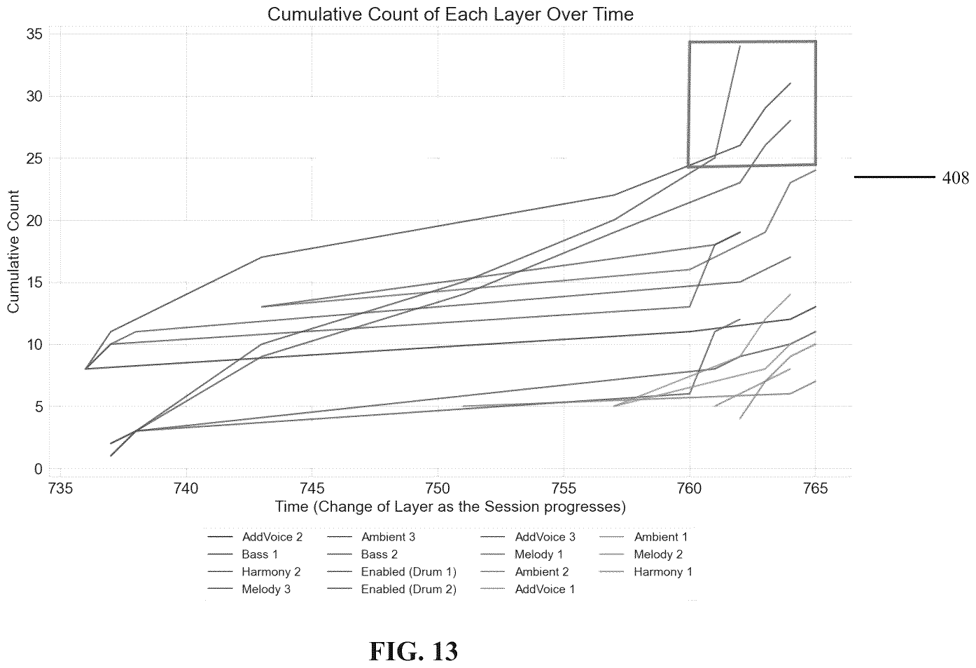

As the sessions progressed and participants became more familiar with uCue, they began to favor new layer combinations. Less than half (3/7, 42.85%) transitioned from soothing melodies to faster-paced songs. The Harmony and Ambient layers were often played in conjunction with the song “Wheels on the Bus” to make it soothing. Several participants (3/7) customized the “Ants Go Marching” by adding drums and other layers, indicating a preference for personalization and active engagement. Based on the data from the logs of a few participants (for C5 and C6), the drums and bass line were added to all four songs to make the environment more energetic. FIGS. 12 and 13 show the time to layer distribution for C5 for two songs.

### Individual Musical Preferences

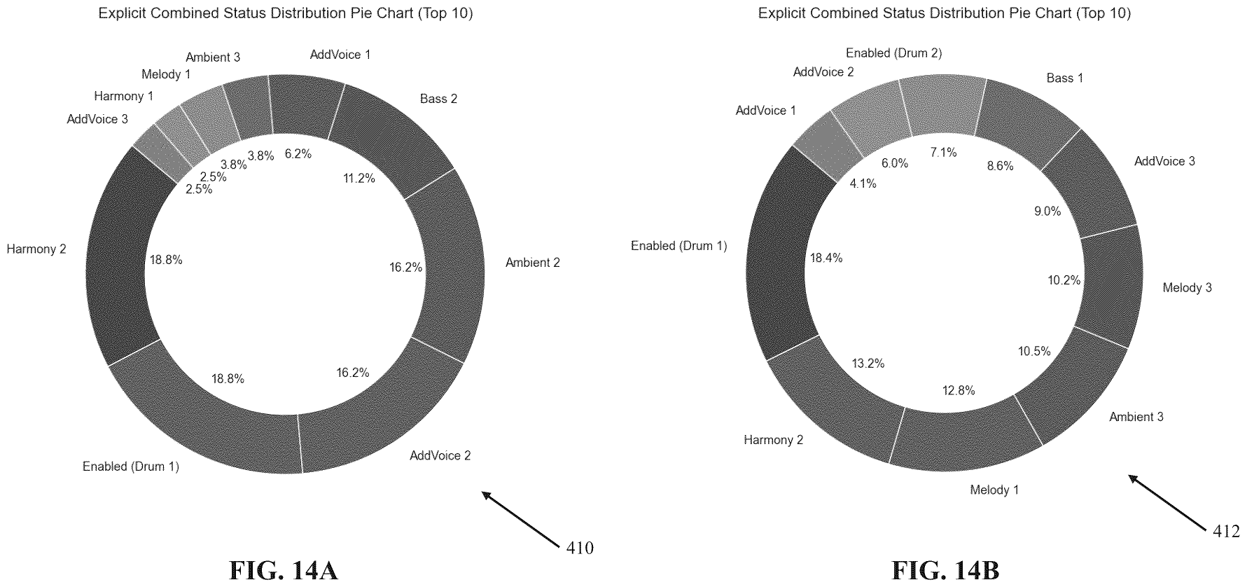

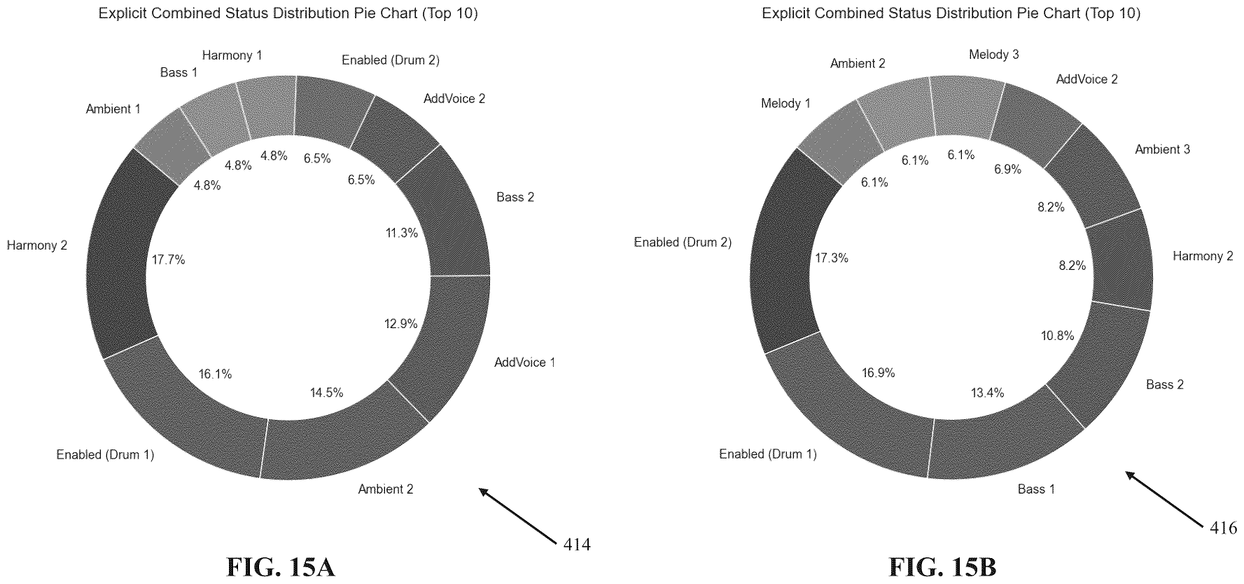

Based on the wide range of genres, tempos, and instruments uCue offers, musical preferences resonated differently with each individual. Unique music preferences were compared across participants to find patterns, outliers, and insights through the subsequent graphs and analyses. FIGS. 14A and 15A illustrate the distinct musical choices of participants C3 and C5 for “Ants go Marching” and FIGS. 14B and 15B for “Wheels on the Bus,” with C3 preferring soft, harmonious layers and C5 opting for upbeat, fast-paced layers with drums.

### Reactions to Ambient Sound Layers.

uCue allowed the addition of ambient sounds; being played at an average of 18-20% per session. Some children enjoyed this addition. For example, after adding the sound of running water to “Twinkle, Twinkle,” C6 commented that “It feels like I am underwater”. In contrast, C4 seemed to become upset by the same sound and briefly exited the session. P4 offered that one possible explanation was an aversion to sounds of water. P4 explained, “He hated the sound of ocean waves since the time he went too near the ocean for the first time”. Similarly, most participants were indifferent to the sound of a train, though C2 had a negative reaction, as P2 noted in the post-session interview. These observations suggest a range of potential responses to different ambient sounds.

### Participant Reactions and Engagement Trends in Video Sessions.

Table 3 (above) summarizes participant's reactions across the seven sessions. We analyzed the videos by tagging actions representing key engagement features, such as enthusiastically reacting to music (i.e., by smiling, or making other positive vocalizations), exhibiting energy by moving their hands or physically reacting by dancing, rocking, singing or tapping along. Each child is represented with “C” (e.g., C1 is the first). During these sessions, it was observed that physical movement played a big role in children's engagement with music. They danced, tapped, waved, and mimicked percussion tracks. Some sang along (C3) and tapped their feet (C5, C6), while others felt compelled to move when they heard certain sounds and rhythms. Furthermore, the interactive device created an experience where children could freely explore and express their creativity or mood. For example, C7, changed a song until it made him “sleepy.” Contrarily, C2 was not interested in interacting with uCue for long and preferred to engage with other devices in the Sensory Room. Unlike some of the other participants, C4 relied heavily on non-verbal forms of communication, such as making noises and physical movements, in addition to using a few words here and there. It was apparent that he initially felt overwhelmed when he entered the room and did not want to engage (i.e., in these cases the research team moved on to the parent debrief interview). P4 mentioned that visuals were a big and important component for him when enjoying music, which uCue did not support, and any unpredictable or unfamiliar sounds tended to upset him (i.e., C4).

### Post Study Interview Analysis

The post-session interviews with parents and guardians yielded valuable insights for understanding each child's behavior. It provided context around their child's reactions and make observations about their child's experience, which may not have been evident to single session observers.

Parents' Perceptions of uCue and Children's Engagement

Parental intervention was encouraged during the sessions to aid communication and foster engagement. Parents often rephrased researcher instructions or provided light encouragement, which helped children navigate the system. In response to questions about their child's experience with uCue, parents highlighted a sense of autonomy that their children appeared to experience while using the system. P6 noted, “My child was excited to explore the device and try different sounds,” suggesting a sense of independent exploration. P7 emphasized the interactive nature, observing, “ . . . liked changing colors and playing with the buttons. It showed they were genuinely engaged and curious.”

Parents also appreciated the variety of songs and identified specific moments where autonomy was evident. For example, P2 noted their child's preference for familiar songs, stating, “He likes things that are familiar . . . he really liked that he could hit the buttons,” indicating how the system allowed the child to make choices aligned with personal preferences. Similarly, P3 observed, “My child's interest in the device changed with different songs,” further supporting the idea that children could exercise control over their listening experience.

The feedback also revealed that the ability to switch between musical layers and experiment with different sounds provided opportunities for decision-making and self-expression. P7 observed, “The rhythmic beats seemed to stimulate their movements and energy levels,” while P6 noted moments of both joy and frustration, “My child smiled and laughed while using the device but also got frustrated when a song stopped abruptly.” These moments highlight the children's active participation in shaping their interaction with the music, reflecting a level of autonomy even when challenges arose. Overall, the uCue system's design-combining interactive layers with modular music-enabled children to make choices about how they interacted with the music. While fluctuations in interest and engagement were observed, these moments of decision-making provided insights into how children could exercise autonomy within the uCue framework.

### DISCUSSION

The findings from our study suggest the potential value, enjoyment, and access that children with ASD experience when personalizing music to their taste. Here we address our research questions, discuss how the collected data may be incorporated into music therapy and composition.

### Interactive Music Making, Autonomy, & Enjoyment

Regarding RQ1 addressing the effectiveness of uCue as a tool for measuring listener preferences, our results suggest uCue's data logs can help generate potential inferences about global and individual preferences. For example, preferences for soothing and energetic music. Such inferences could have utility-particularly with higher n-values and more fine-grain analysis. Our RQ2 explored whether engagement with uCue could facilitate emotional experience and enhance enjoyment of music for children with ASD. The findings from Table 3 highlight that most participants enthusiastically reacted to the music and all the participants exhibited a noticeable mood change. These results underscore the potential of uCue to provide an enjoyable and emotionally expressive platform for children with ASD, fostering a deeper connection to music. Regarding RQ3, uCue fostered autonomy by enabling children with ASD to actively shape their musical experiences. Rather than passively listening, children made decisions about adding, modifying, or layering musical components, allowing them to personalize their engagement. This autonomy was reflected in their exploration of favored sounds, selection of familiar tunes, and experimentation with rhythmic or melodic elements. Parental feedback highlighted that the ability to control and customize the music heightened children's engagement and encouraged decision-making. By empowering children to take an active role, uCue supported autonomy in both musical expression and listening experiences.

This stage focused on exploratory interactions with uCue rather than comparative analysis, with the objective to demonstrate the potential of uCue as an interactive music system and data collection system and to document how participants engage with the system.

While the uCue system provides numerous button and musical combinations, Table 3 shows that most participants (5/7) effectively navigated the musical layers and selected their preferred combinations, demonstrating the system's usability. During the sessions, the research team observed that the patterned musical layer to lights mapping (Table 1) appeared to aid participants by providing clear visual cues that helped them track active layers and maintain focus. This visual mapping appeared to reduce sensory overload and facilitated smoother interactions with the system. Additionally, incorporating ambient sounds made the environment more immersive and enjoyable for some, though it triggered negative reactions in others, underscoring uCue's potential for studying listening sensitivities and individual sound preferences.

### Application in Musical Therapy

One dimension of uCue is its potential to support socio-emotional learning objectives commonly emphasized in music therapy. uCue-based activities may encourage participants to communicate their feelings and reflect specific moods through their musical creations. To support these goals, updates to uCue's playback settings (Section 6.3), protocol and moderator guide, and implemented assistive communication tools such as PECS cards improved data collection, fostered communication, and enhanced participant engagement.

Qualitative observations during the sessions indicated that even young participants with limited verbal communication abilities were able to engage in the activities and communicate their preferences, such as indicating when they liked a particular song or sound or when a song made them feel calm or energetic. These observations suggest the potential for uCue-based activities to support socio-emotional learning goals, though further systematic evaluation is required. While we do not yet have quantitative evidence to substantiate these claims, the qualitative feedback from participants and their caregivers underscores the promise of refining uCue to further support socio-emotional learning and social interaction.

### Musical Composition Challenges

Since its inception, one research goal has been to leverage interaction data to optimize uCue for its audience. Though issues like listener preferences for timbres, textures, song length, repetition, and ambient sounds need further exploration, current findings have significantly influenced uCue's playback and composition. In exemplary embodiments, songs started with a calm default track that users could adjust to heighten stimulation. Not all users prefer a slow tempo, so embodiments may include options for variable tempos to enhance user experience. Various musical introductions were tested, revealing that longer ones hinder users' timing for participation. Clear introductions of song identity and participation cues were found to be useful.

In the hands of its users, uCue is a musical instrument that enables them to exercise creative control while listening. This fact was shown clearly by one child (C2), who began the session clumsily using his palm to press multiple buttons simultaneously. Before long, and to the astonishment of his parent, the child began using his fingers to press buttons, quickly developing the same auditory-directed motor control musicians use to learn traditional instruments. Anecdotes like the one above demonstrate the potential of uCue to provide authentic and rewarding musical experiences.

### Design Variations

### Multisensory Engagement

Parents stressed the significance of offering age-appropriate music and casing design that aligns with a child's developmental stage, ensuring it resonates with their cognitive and emotional capacity. Using an interface that links different colors to different music layers created an exciting way for children to interact with music. Flashing lights synchronized with the music may improve the visual appeal, captivating children's attention and creating a multisensory experience that supports engagement.

### Plan for Diverse Track Selection and Feedback

Offering a diverse array of music tracks to accommodate varying preferences among young children is desirable. The tracks on uCue were met with mixed reactions. Some thought the tracks were perfect for the age group, while others desired more advanced material. A culturally diverse and extensive library may beneficially cater to the musical preferences of children, particularly older children and those with different cultural backgrounds. A set of user preference buttons may be incorporated on the audio playback device, including presets for individual users, initial track options catering to volume preferences (e.g., soft vs. loud), and buttons allowing users to provide real-time feedback about their liking or disliking of specific sounds and arrangements.

### Include Tailored Musical Variability

Based on the parents' perceptions of their children's experience with uCue and findings from logs, embodiments that facilitate dynamic and tailored musical variability, such as adapting the music's volume, tempo, style, or complexity via direct interaction with the audio channels or based on the child's engagement level, may be desirable.

### CONCLUSION

uCue provides a platform for engaging children with ASD in formative, participatory musical experiences and generating user data that can address various questions about listening preferences, emotional regulation, and social-skill development. As applied research designed to utilize the potential for HCl to inform scholarly and creative activity, this project opens new lines of inquiry within the field of autism studies. It provides a tool that can be adapted for use in a variety of listening contexts, including home, libraries, schools, hospitals, therapy sessions, and other social settings. By employing an interdisciplinary approach to addressing a societal problem, we explore a potential solution that integrates computer science, music theory, and composition, highlighting the mutually enriching nature of such collaborative efforts. uCue empowers its users to become composers as they listen and to express emotions as they experience sounds.

### Example 2

### Introduction

Interactive dashboards are visual tools that enable users to explore and manipulate data, uncovering complex patterns and insights, such as user behavior trends in web analytics, that may be missed by traditional, static visualizations. They have proven effective in fields such as business intelligence, big data, healthcare, and education by enabling users to make data-driven decisions and understand complex data at a glance. Despite their success in these areas, the application of interactive dashboards in music composition remains underexplored. An interactive dashboard for music composition may offer composers new ways to visualize and analyze musical elements, helping them create pieces that may be tailored to the needs of specific audiences, such as children with ASD. This approach may facilitate a deeper understanding of how different musical components interact, thereby enhancing the creative process and improving composition outcomes.

The therapeutic potential of music for individuals with Autism Spectrum Disorder (ASD) has been widely recognized, offering benefits ranging from improved social interaction to increased emotional regulation. Music therapy has been widely used to support the development of communication, social, and emotional skills in children with ASD, often involving interactive musical activities tailored to the child's needs. Traditional music therapy methods, however, often rely on qualitative assessments that can miss subtle interaction patterns that can provide valuable insights for composing therapeutic music. To address this, uCue was introduced, a musical interface designed to empower children with ASD to modify songs by adding and subtracting various musical layers. uCue logs interaction data, capturing which musical layers were added or subtracted by the user. The logs may gather valuable interaction data and provide insights about global musical preferences, including preferences and dislikes for specific musical styles, layer combinations, and energy levels. This interaction data was acquired and analyzed to create interactive visuals aimed to assist musical composers to identify necessary musical patterns and refine their composition.

However, music composers may face barriers engaging with interactive music data due to limited knowledge of information visualization, which could hinder their ability to utilize these tools effectively. Visualization literacy, the skill of interpreting visual data representations, is not widely prevalent, including among composers, impacting their use of visual analytics. Given this challenge, there is a need for accessible tools that simplify data interpretation, enabling composers to explore new creative possibilities and enhance music composition for therapy sessions. Despite the potential benefits, integrating interactive visualizations into music composition is still nascent, and existing tools often do not meet the specific needs of music composition as they lack the adaptability to represent the layered, temporal, and interactive nature of music effectively. This example describes an interactive dashboard, “MusicVis”—composed of various visualizations to enhance music composition for therapeutic interventions, utilizing the output of aspects of an exemplary system as described herein. It should be understood that the processor or computer referenced herein may be a same processor/computer configured as the DMC as referenced herein, or may be a different computer/processor that may be programmed with additional machine-readable instructions used by composers, songwriters, and audio engineers for creating and finishing musical compositions.

### Music Therapy for ASD

Music therapy is recognized as a valuable tool in working with children diagnosed with ASD. The therapeutic use of music helps in improving communication, social interaction, and emotional regulation.

Insights into ASD-specific musical preferences informed the development of Music Vis, guiding the design to tailor visualizations that emphasize these critical musical elements. Music Vis, through its specialized visualizations, was designed to help music composers discover and understand these insights. By visually highlighting critical musical elements, the dashboard may facilitate composers' ability to align their musical compositions with therapeutic goals, ensuring that the created music resonates effectively with the unique sensory profiles of individuals with ASD.

### Music Composition: Technology and Confidence

Music Composition is a creative process. The integration of technology into music composition has opened up new possibilities for enhancing the creative process. Technologies such as digital musical instruments, software-based composition tools, and interactive interfaces have been leveraged to tailor the music-making process to the specific needs of individuals, particularly children with ASD, offering them opportunities for active participation and creative expression. These tools not only provide composers with a broader palette of sounds and structures to work with but also facilitate experimentation and iteration, allowing for rapid prototyping and exploration of new musical ideas. Furthermore, interactive interfaces and new musical expression technologies make it possible to create adaptive and responsive compositions that can evolve based on user interaction, thereby supporting a more dynamic and inclusive approach to composition. This adaptability is desirable for creating music that is not only aesthetically rich but also accessible and engaging for diverse audiences, including those with special needs.

### Visual-Interactive Dashboards for Creative Purposes

Visual-interactive dashboards are increasingly being used in various fields to organize, present, and intuitively interact with complex data. In both creative and therapeutic contexts, these dashboards may serve as essential tools for tracking progress, identifying patterns, and supporting decision-making. In healthcare, dashboards monitor patient data to identify trends, predict outcomes, and assist clinicians in making informed decisions, such as analyzing cancer cohorts to personalize treatments. In education, they track learning outcomes to support educators in tailoring instructional strategies and interventions to improve student performance. In creative arts therapies, these tools aid therapists and clients by facilitating the creative process, enabling the exploration of emotions, and guiding therapeutic interventions through visual feedback and analysis. When applied to music composition, these dashboards may offer music composers and therapists a dynamic platform to visualize and interact with musical data, enhancing the creative process and therapeutic efficacy.

In the context of music therapy, visual-interactive dashboards may be instrumental in identifying and analyzing musical patterns created by users of interactive music technology. The patterns in this interaction data might include the frequency and duration the listener preferred certain musical elements, the transitions between different sections of a composition, or the overall structure of a musical piece. By visualizing these patterns, composers may gain insight into the child's interaction with the music and tailor their therapeutic approach accordingly. The dashboards may allow one to compare different sessions or participants, and may provide a deeper understanding of how music therapy affects each individual.

While dashboards have shown considerable potential in creative and therapeutic settings, there is still a significant gap in research focused on their application in music composition for children with ASD. Few studies have explored how these tools can support the creative aspects of music therapy, such as composition and improvisation. Additionally, there is a desire for research on the usability and accessibility of these dashboards for composers to ensure they can fully engage with the tool and benefit from its features.

### Music Visualization

Interactive visual dashboards may enhance music composition by providing composers with intuitive interfaces to explore song structures. These systems often incorporate modeling techniques that visually represent rhythmic patterns. More broadly, visualization techniques in music utilize visual representations to depict elements such as rhythm, melody, harmony, and dynamics-simplifying complex musical concepts and making them more accessible, particularly for educational and creative purposes. However, most existing techniques focus on isolated musical characteristics, such as rhythm or harmony, rather than integrating these elements comprehensively, especially in therapeutic contexts. Research shows that music may enhance engagement, support learning, and facilitate emotional expression in therapeutic contexts. Visualization techniques may help composers assess a child's musical preference, guiding the composition process.

Research on the use of visualizations in interactive music composition is still in its early stages, but initial findings suggest that they may be an effective tool for enhancing the compositional process. They may act as a medium to offer a visual language that allows composers to express musical ideas more intuitively. This may make it easier for composers to conceptualize and manipulate musical elements, facilitating a deeper exploration of creative possibilities.

### Visualizations in the Dashboard

The dashboard may incorporate a range of visualizations designed to analyze both interaction data collected from participants and musical data. These visualizations may be categorized into two primary groups: Interaction Data and Musical Data. All the visualizations were made using plotly, which provided interactivity, and were deployed in the form of a dashboard via dash. The design of these visualizations was directly influenced by the challenges faced during the music composition process, so that the tools provided may effectively support the composers in their work.

### Features of the Dashboard

The visual dashboard developed for this study incorporates several key features aimed at enhancing the analysis and application of interactive music sessions for children with ASD.

### Audio and Comparative Tools

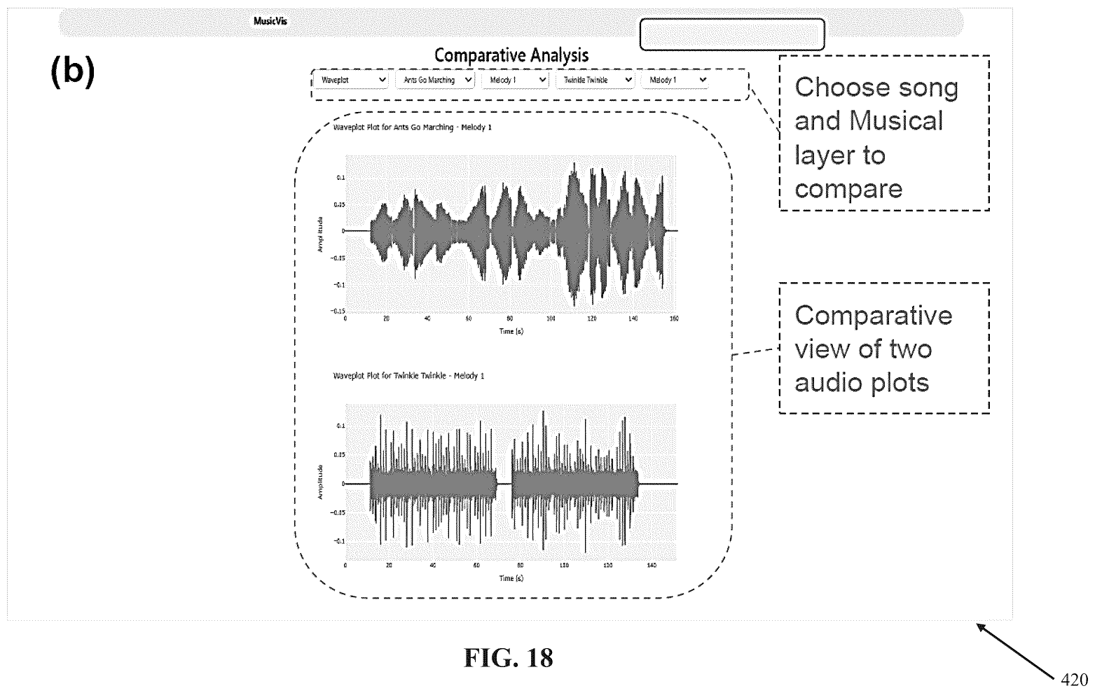

The system may archive audio data, allowing for comparative analysis of musical compositions. The archived audio data may be accessible through the Comparative Analysis section, which may enable therapists and composers to compare different musical compositions and their effects. This feature may support the creation of a comprehensive, modular music library tailored for therapeutic use with children with ASD by leveraging historical data to refine personalized interventions. FIG. 18 depicts an overview of the Comparative Analysis section to compare the audio composition of different audios.

### Cohort Analysis and Dynamic Visual Representation of Session Data

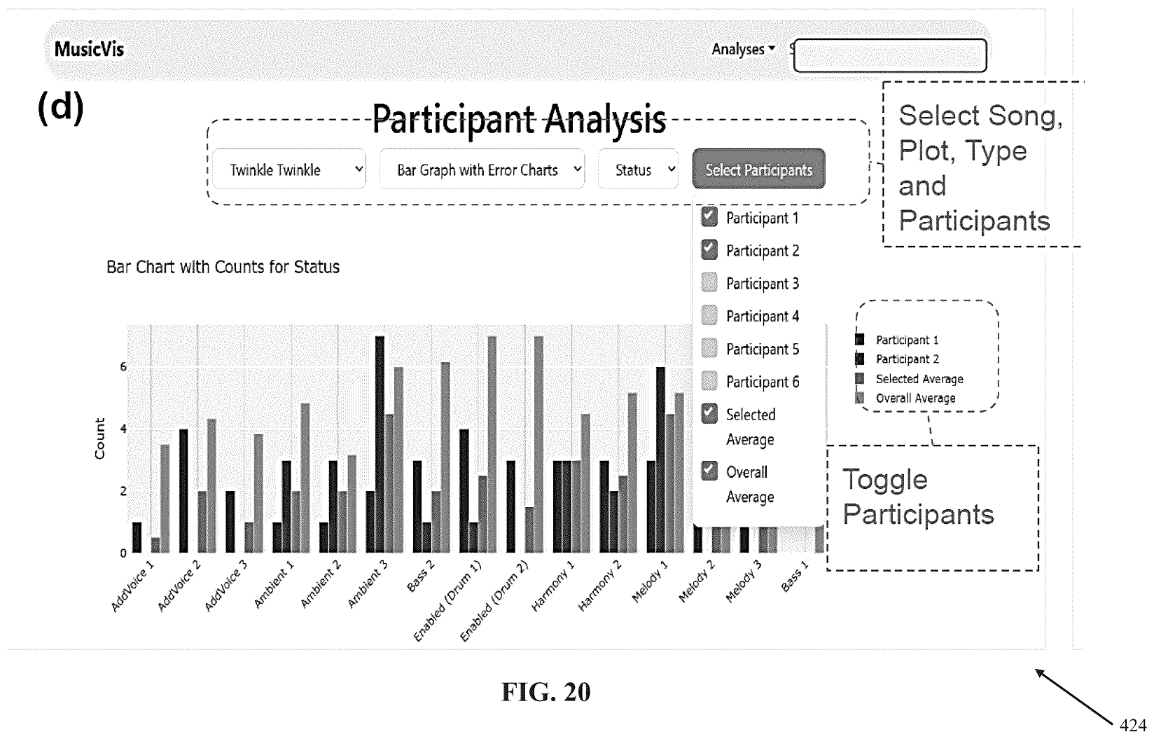

The dashboard may provide real-time visualization of interactive music sessions, allowing therapists and composers to observe how children with ASD engage with different musical elements. The “Participant Analysis” section may enable users to analyze data for individual participants or cohorts, offering flexibility in understanding engagement patterns across multiple sessions. From the visualizations, users may identify trends and insights, enhancing data interpretation and decision-making. FIG. 20 depicts the Participant Analysis section of the Dashboard.

### Full Session Diagram

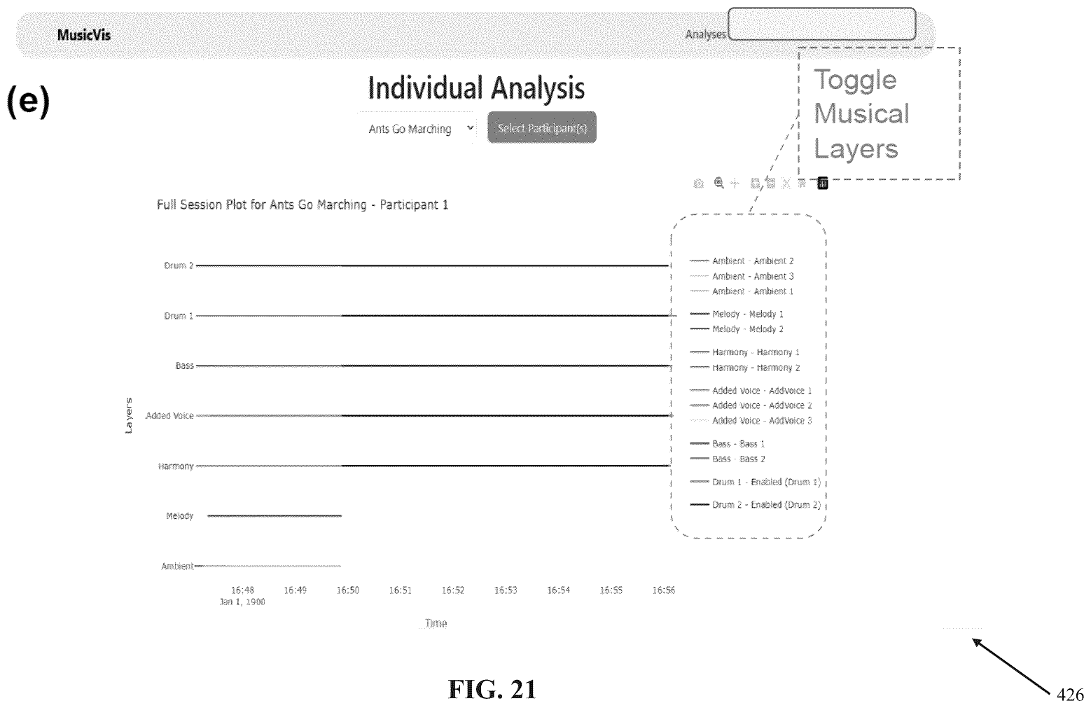

The Full Session Diagram tracks the activation and deactivation of musical layers (e.g., Melody, Harmony, Bass) throughout therapy sessions, providing a clear timeline to understand how different elements affect participant engagement and responses. This tool may be desirable for analyzing music patterns and optimizing interventions based on temporal data. FIG. 21 depicts the interface to analyze individual sessions.

### Interaction and Musical Data

Overall, the results of this usability study suggest that interaction data and the visual analytics dashboard may potentially assist in guiding musical composition. While the dashboard provided participants with musical data, such as spectral energy, pitch, and brightness, for experimenting with different elements, participants also considered integrating interaction data was viewed as a valuable addition.

Integrating interaction data into the dashboard enabled participants to understand not just the ‘what’ but also the ‘how’ and ‘why’ of their music choices, leading to more personalized and effective music interventions for children with ASD. It gave composers a contextual understanding of how various elements combined to create a cohesive auditory experience that aligned with listener preferences. Participants highlighted that the interaction data showed which layers were the most used and how these layers interacted rhythmically and dynamically over time. For example, using a Sankey diagram to visualize transitions helped reveal frequency, pitch, and amplitude patterns. This data-driven approach facilitated more informed compositions by allowing experimentation based on visual feedback.

Although musical data provided insight into sound properties, such as identifying layers with higher spectral energy or lower brightness, participants valued interaction data as crucial for decision-making. Without it, their understanding remained static and less connected to the overall composition flow. Thus, interaction data may enrich their understanding of how musical elements coalesced, and may make it a useful component for creating new compositions that are both complex and potentially therapeutically effective.

### Exemplary Computer and Processors Hardware

As used herein, a processor is a hardware circuit having elements structured and arranged to perform one or more processing functions, typically various data processing functions. Although discrete logic components could be used, the examples utilize components forming a programmable central processing unit (CPU). A processor, for example, includes or is part of one or more integrated circuit (IC) chips incorporating the electronic elements to perform the functions of the CPU. The processor may be based on any known or available microprocessor architecture, such as a Reduced Instruction Set Computing (RISC) using an ARM architecture, for example. Of course, other processor circuitry may be used to form the CPU or processor hardware in. The processor can include one microprocessor or a multi-processor architecture. A digital signal processor (DSP) or field-programmable gate array (FPGA) could be suitable replacements for the processor, but may consume more power with added complexity.

Each system processor includes one or more central processing units (CPUs) and local memory corresponding to each CPU, as well as shared memory available to any CPU. An operating system (OS) executed by the system processors manages the various jobs and applications currently running to perform appropriate processing. The OS also provides a system management facility (SMF) and open exit points for managing the operation of the system and the various jobs and applications currently running. The applicable processor executes programming or instructions to configure a system to perform various operations.

A computer type user terminal device, such as a PC, for example, can similarly include a data communication interface CPU, main memory and one or more mass storage devices for storing user data and the various executable programs. The various types of user terminal devices will also include various user input and output elements. A computer, for example, may include a keyboard and a cursor control/selection device such as a mouse, trackball, or touchpad; and a display for visual outputs.

In the examples above, the processor can include a memory. The memory may include a flash memory (non-volatile or persistent storage), a read-only memory (ROM), and a random access memory (RAM) (volatile storage). The RAM serves as short term storage for instructions and data being handled by the processor(s) as a working data processing memory. The flash memory typically provides longer term storage.

Of course, other storage devices or configurations may be added to or substituted for those in the example. Such other storage devices may be implemented using any type of storage medium having computer or processor readable instructions or programming stored therein and may include, for example, any or all of the tangible memory of the computers, processors or the like, or associated modules.

Hence, a machine-readable medium or a computer-readable medium may take many forms of tangible storage medium. Non-volatile storage media include, for example, optical or magnetic disks, such as any of the storage devices in any computer(s) or the like, such as may be used to implement the client device, media gateway, transcoder, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

According to exemplary embodiments of the present disclosure the one or more processors and control circuits can include one or more of any known general purpose processor or integrated circuit such as a central processing unit (CPU), microprocessor, field programmable gate array (FPGA), Application Specific Integrated Circuit (ASIC), Digital Signal Processor (DSP), or other suitable programmable processing or computing device or circuit as desired that is specially programmed to perform operations for achieving the results of the exemplary embodiments described herein. The processor(s) can be configured to include and perform features of the exemplary embodiments of the present disclosure. The features can be performed through program code encoded or recorded on the processor(s), or stored in a non-volatile memory device, such as Read-Only Memory (ROM), erasable programmable read-only memory (EPROM), or other suitable memory device or circuit as desired. Accordingly, such computer programs can represent controllers of the computing device.

In another exemplary embodiment, the program code can be provided in a computer program product having a non-transitory computer readable medium, such as Magnetic Storage Media (e.g. hard disks, floppy discs, or magnetic tape), optical media (e.g., any type of compact disc (CD), or any type of digital video disc (DVD), or other compatible non-volatile memory device as desired) and downloaded to the processor(s) for execution as desired, when the non-transitory computer readable medium is placed in communicable contact with the processor(s).

The one or more processors can be included in a computing system that is configured with components such as memory, a hard drive, an input/output (I/O) interface, a communication interface, a display and any other suitable component as desired. The exemplary computing device can also include a communications interface. The communications interface can be configured to allow software and data to be transferred between the computing device and external devices. Exemplary communications interfaces can include a modem, a network interface (e.g., an Ethernet card), a communications port, a PCMCIA slot and card, or any other suitable network communication interface as desired. Software and data transferred via the communications interface can be in the form of signals, which can be electronic, electromagnetic, optical, or other signals as will be apparent to persons having skill in the relevant art. The signals can travel via a communications path, which can be configured to carry the signals and can be implemented using wire, cable, fiber optics, a phone line, a cellular phone link, a radio frequency link, or any other suitable communication link as desired.

Where the present disclosure is implemented using programming or software, the programming or software can be stored in a computer program product or non-transitory computer readable medium and loaded into the computing device using a removable storage drive or communications interface. In an exemplary embodiment, any computing device disclosed herein can also include a display interface that outputs display signals to a display unit, e.g., LCD screen, plasma screen, LED screen, DLP screen, CRT screen, or any other suitable graphical interface as desired.

Hence, aspects of the methods described herein may be embodied in programming. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through a global information network (e.g. the Internet®) or various other telecommunication networks. Such communications, for example, may enable loading of the software from one computer or processor into another, for example, from a management server or host computer, such as a proxy server, for example, into a mainframe platform that will execute the various jobs. Thus, another type of media that may bear the software elements includes optical, electrical and electromagnetic waves, such as used across physical interfaces between local devices, through wired and optical landline networks and over various air-links. As used herein, unless restricted to non-transitory, tangible “storage” media, terms such as computer or machine “readable medium” refer to any medium that participates in providing instructions to a processor for execution.

Non-volatile storage media include, for example, optical or magnetic disks, such as any of the storage devices in any computer(s) or the like, such as may be used to hold datasets and programs for enterprise applications. Volatile storage media include dynamic memory, such as main memory of such a computer platform. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge or any other medium from which a computer can read programming code and/or data. Many of these forms of computer readable media may be involved in carrying one or more sequences of one or more instructions to a processor for execution.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

What is claimed is:

1. An interactive audio control device comprising:
a user interface comprising a plurality of selectable user input elements, each selectable user input element comprising an audio playback control corresponding to (i) a predetermined audio layer of a selected multi-layer musical track or (ii) a playback operation, each selectable user input element corresponding to a predetermined audio layer configured to select from a plurality of playback modes corresponding to the predetermined audio layer and to provide a visual indication corresponding to the selected playback mode.

2. The interactive audio control device of claim 1, wherein the interactive audio control device comprises a housing, the user interface comprises a keypad disposed within the housing, and each of at least a subset of the plurality of selectable user input elements comprises a depressible button disposed on the keypad.

3. The interactive audio control device of claim 2, wherein each depressible button corresponding to a predetermined audio layer is configured to permit a device user to sequentially step through the plurality of playback modes by repeatedly pressing the depressible button.

4. The interactive audio control device of claim 3, wherein the visual indication comprises a different color corresponding to the selected playback mode.

5. The interactive audio control device of claim 4, wherein each depressible button comprises a plurality of LEDs disposed beneath a translucent cover, the different color corresponding to illumination of a different LED or different combination of LEDs corresponding to each of the selected playback modes.

6. The interactive audio control device of claim 1, further comprising:
a receiver for receiving audio layer playback mode data; and
a transmitter for transmitting audio playback control output data, the audio playback control output data comprising one or more device user inputs.

7. A system for evaluating interactivity of a device user with music audio during an evaluation session, the system comprising:
the interactive audio control device of claim 6;
one or more audio output devices;
at least one device monitor-controller (DMC), the DMC comprising:
at least one memory configured to store (i) data received from the transmitter of the interactive audio control device and (ii) an audio library comprising audio data for a plurality of multi-layer musical tracks;
at least one DMC user interface;
at least one display; and
a communication link configured to exchange information with the interactive audio control device and the one or more audio output devices;
at least one processor in communication with the communication link, the at least one memory, and the at least one display, the at least one processor configured to:
generate, via the at least one display, a graphical user interface (GUI) that displays settings of the interactive audio control device, a plurality of device monitor-selectable user input elements and a menu of the plurality of multi-layer musical tracks;
receive, via at least one of the interactive audio control device via the communication link or via the at least one DMC user interface, one or more setting selection inputs, each setting selection input comprising a device user input from the interactive audio control device or a corresponding DMC user input from the DMC user interface, each setting selection input selected from the group consisting of: a multi-layer musical track selection, a playback mode selection, and a playback operation selection;
output, to the interactive audio control device via the communication link, audio layer playback mode data based on the received one or more setting selection inputs;
transmit, to the one or more audio output devices via the communication link, playback audio based on the received one or more setting selection inputs;
log interactivity data corresponding to each setting selection input; and
store, in the at least one memory, the interactivity data.

8. The system of claim 7, wherein:
the DMC user interface GUI includes an icon corresponding to each button of the interactive audio control device.

9. The system of claim 8, wherein each icon has a plurality of display formats, each corresponding to a corresponding visual indication of the interactive audio control device.

10. The system of claim 9, wherein each display format comprises an icon color corresponding to the color of its corresponding button of the interactive audio control device, and wherein providing the visual indication comprises changing the color of one or more buttons and each corresponding icon based on the selected playback mode.

11. The system of claim 7, wherein each of the plurality of audio layers comprises a plurality of playback modes, the plurality of playback modes comprising one or more distinct playback modes and a mute mode.

12. The system of claim 7, wherein the interactivity data comprises timestamp data, audio layer data, and playback mode data.

13. The system of claim 12, wherein logging the interactivity data comprises:
recording, in one or more data files:
a timestamp for each setting selection input received by the at least one processor;
a title of the multi-layer musical track being output as playback audio when each setting selection input is received; and
the playback mode of each layer of the multi-layer musical track being output as playback audio when each setting selection input is received.

14. The system of claim 7, wherein the at least one processor is further configured to generate one or more visual data representations for evaluation, each of the one or more visual data representations comprising at least one of the interactivity data or the audio data.

15. A method for evaluating interactivity of a device user with music audio during an evaluation session comprising:
providing a system for evaluating interactivity of a device user with music audio during an evaluation session comprising:
an interactive audio control device comprising a user interface comprising a plurality of selectable user input elements, each selectable user input element comprising an audio playback control corresponding to (i) a predetermined audio layer of a selected multi-layer musical track or (ii) a playback operation, each selectable user input element corresponding to a predetermined audio layer configured to select from a plurality of playback modes corresponding to the predetermined audio layer and to provide a visual indication corresponding to the selected playback mode;
one or more audio output devices;
at least one device monitor-controller (DMC), the DMC comprising:
at least one memory configured to store (i) data received from a transmitter of the interactive audio control device and (ii) an audio library comprising audio data for a plurality of multi-layer musical tracks;
at least one DMC user interface;
at least one display;
a communication link configured to exchange information with the interactive audio control device and the one or more audio output devices;
at least one processor in communication with the communication link, the at least one memory, and the at least one display;
generating, by the at least one processor, via the at least one display, a graphical user interface (GUI) that displays settings of the interactive audio control device, a plurality of device monitor-selectable user input elements and a menu of the plurality of multi-layer musical tracks;
receiving, by the at least one processor, via at least one of the interactive audio control device via the communication link or the at least one DMC user interface, one or more setting selection inputs, each setting selection input comprising a device user input from the interactive audio control device or a corresponding DMC user input from the DMC user interface, each setting selection input selected from the group consisting of: a multi-layer musical track selection, a playback mode selection, and a playback operation selection;
outputting, to the interactive audio control device via the communication link, audio layer playback mode data based on the received one or more setting selection inputs;
transmitting, to the one or more audio output devices via the communication link, playback audio based on the received one or more setting selection inputs;
logging, by the at least one processor, interactivity data corresponding to each setting selection input;
storing in the at least one memory, by the at least one processor, the interactivity data; and
generating, by the at least one processor, an evaluation output comprising at least one of the interactivity data or the audio data.

16. The method of claim 15, wherein the evaluation output further comprises one or more visual data representations, each of the visual data representations comprising at least one of the interactivity data or the audio data.

17. The method of claim 16, further comprising:
generating, by the at least one processor, a composer-interactivity interface comprising the one or more visual data representations on the at least one display.

18. A non-transitory computer memory programmed with computer-readable instructions for causing a computer to perform the method steps of:
generating, by at least one processor in communication with the non-transitory computer memory via a communication link, via at least one display, a graphical user interface (GUI) that displays settings of an interactive audio control device, a plurality of device monitor-selectable user input elements and a menu of a plurality of multi-layer musical tracks, each of the multi-layer musical tracks comprising a plurality of audio layers, each audio layer comprising a plurality of playback modes;
receiving, by the at least one processor, via at least one of the interactive audio control device via the communication link or at least one device monitor-controller (DMC) user interface, one or more setting selection inputs, each setting selection input comprising a device user input from the interactive audio control device or a corresponding DMC user input from the DMC user interface, each setting selection input selected from the group consisting of: a multi-layer musical track selection, a playback mode selection, and a playback operation selection;
outputting, by the at least one processor, to the interactive audio control device via the communication link, audio layer playback mode data based on the received one or more setting selections;
transmitting, by the at least one processor, to one or more audio output devices in communication with the communication link, playback audio based on the received one or more setting selections;
logging, by the at least one processor, interactivity data corresponding to each setting selection input;
storing in the non-transitory computer memory, by the at least one processor, the interactivity data; and
generating, by the at least one processor, an evaluation output comprising at least one of the interactivity data or audio data for one or more of the plurality of multi-layer musical tracks.

19. The method of claim 18, wherein the evaluation output further comprises one or more visual data representations, each of the visual data representations comprising at least one of the interactivity data or the audio data.

20. A device monitor-controller (DMC) configured for communication with an interactive audio control device and one or more audio output devices, the DMC comprising:
at least one memory configured to store (i) data received from a transmitter of the interactive audio control device and (ii) an audio library comprising audio data for a plurality of multi-layer musical tracks;
at least one DMC user interface;
at least one display;
a communication link configured to exchange information with the interactive audio control device and the one or more audio output devices;
at least one processor in communication with the communication link, the at least one memory, and the at least one display, the at least one processor configured to:
generate, via the at least one display, a graphical user interface (GUI) that displays settings of the interactive audio control device, a plurality of device monitor-selectable user input elements and a menu of the plurality of multi-layer musical tracks;
receive, via at least one of the interactive audio control device via the communication link or via the at least one DMC user interface, one or more setting selection inputs, each setting selection input comprising a device user input from the interactive audio control device or a corresponding DMC input from the DMC user interface, each setting selection input selected from the group consisting of: a multi-layer musical track selection, a playback mode selection, and a playback operation selection;
output, to the interactive audio control device via the communication link, audio layer playback mode data based on the received one or more setting selection inputs;
transmit, to the one or more audio output devices via the communication link, playback audio based on the received one or more setting selection inputs;
log interactivity data corresponding to each setting selection input; and
store, in the at least one memory, the interactivity data.
